(19) United States
(12) Patent Application Publication
(10) Pub. No.: US 2026/0111108 A1
(43) Pub. Date: Apr. 23, 2026

(54) ELECTRONIC APPARATUS AND CONTROLLING METHOD THEREOF

(71) Applicant: SAMSUNG ELECTRONICS CO., LTD., Suwon-si (KR)

(72) Inventors: Juyeon YOU, Suwon-si (KR); Byunghwa LEE, Suwon-si (KR); Gayeon LEE, Suwon-si (KR); Jinyoung CHOI, Suwon-si (KR)

(73) Assignee: SAMSUNG ELECTRONICS CO., LTD., Suwon-si (KR)

(21) Appl. No.: 19/366,988

(22) Filed: Oct. 23, 2025

Related U.S. Application Data
(63) Continuation of application No. PCT/KR2025/011933, filed on Aug. 7, 2025.

(30) Foreign Application Priority Data
Oct. 21, 2024 (KR) 10-2024-0144227

(51) Int. Cl. G06F 3/0488; A63B 24/00; A63B 71/06; G06F 3/16; G06F 21/32

(52) U.S. Cl. CPC G06F 3/0488; A63B 24/0075; A63B 71/0622; G06F 3/167; G06F 21/32

(57) ABSTRACT

An electronic apparatus includes: a memory configured to store instructions; a microphone; sensor circuitry; a display; and at least one processor including processing circuitry, in which the instructions, when executed by the at least one processor individually or collectively, cause the electronic apparatus to: acquire, through the microphone, audio data including a user's voice, perform a first user authentication based on the audio data, based on determining the first user authentication is successful, control the display to display a first screen including a first body part user interface (UI) corresponding to an authenticated user, acquire, through the sensor circuitry, touch data including a user touch while the first screen is displayed, perform second user authentication based on the touch data, and based on determining the second user authentication is successful, control the display to display a second screen for exercise content.

### CROSS-REFERENCE TO RELATED APPLICATION

This application is a bypass continuation of International Application No. PCT/KR2025/011933, filed on Aug. 7, 2025, which is based on and claims priority to Korean Patent Application No. 10-2024-0144227, filed on Oct. 21, 2024, in the Korean Intellectual Property Office, the disclosures of which are incorporated by reference herein in their entireties.

### BACKGROUND

### 1. Field

The disclosure relates to an electronic apparatus and a controlling method thereof, and more particularly, to an electronic apparatus providing exercise guidance information including exercise content and a controlling method thereof.

### 2. Description of Related Art

Exercise content and exercise guidance information may be output through a display. A user may follow a guided posture while watching the exercise content. It may be inconvenient for the user to follow the posture displayed on the display. Depending on the difficulty of the posture provided in the exercise content, some users may have a problem in not being able to easily follow the posture.

When the exercise posture is followed incorrectly, there may be a risk of injury depending on the type of exercise. The exercise posture may partially differ depending on a user's physical condition such as height, weight, etc. The exercise posture may vary depending on the user's height, weight, muscle mass, gender, flexibility, etc. The risk of injury may occur when a consistent posture is required.

When the user changes to the next posture while not being able to follow a current exercise posture, the user may not feel satisfied with the exercise content or may not be able to concentrate.

### SUMMARY

Provided are an electronic apparatus that provides a body part user interface (UI) for guiding exercise content by considering a user's physical condition, and a controlling method thereof.

According to an aspect of the disclosure, an electronic apparatus includes: a memory configured to store instructions; a microphone; a sensor; a display; and at least one processor including processing circuitry, in which the instructions, when executed by the at least one processor individually or collectively, cause the electronic apparatus to: acquire, through the microphone, audio data including a voice of a user, perform a first user authentication based on the audio data, based on identifying that the first user authentication is successful, control the display to display a first screen including a first body part user interface (UI) corresponding to an authenticated user, acquire, through the sensor, touch data including a user touch while the first screen is displayed, perform second user authentication based on the touch data, and based on identifying that the second user authentication is successful, control the display to display a second screen for exercise content.

The instructions, when executed by the at least one processor individually or collectively, cause the electronic apparatus to: acquire a first difference value between the audio data acquired through the microphone and registered voice data stored in the memory, and identify that the first user authentication is successful based on the first difference value being less than a first threshold value.

The instructions, when executed by the at least one processor individually or collectively, cause the electronic apparatus to: based on identifying that the first user authentication is successful, acquire body part information corresponding to the authenticated user, identify a preset first target part among a plurality of body parts included in the body part information, generate the first body part UI based on the preset first target part, generate the first screen including the first body part UI, and control the display to display the first screen.

The instructions when executed by the at least one processor individually or collectively, cause the electronic apparatus to: based on the first screen being displayed, activate the sensor.

The instructions when executed by the at least one processor individually or collectively, cause the electronic apparatus to: acquire a second difference value between the touch data acquired through the sensor and registered touch data stored in the memory, and identify that the second user authentication is successful based on the second difference value being less than a second threshold value.

The instructions, when executed by the at least one processor individually or collectively, cause the electronic apparatus to: based on identifying that the second user authentication is successful, acquire user information corresponding to the authenticated user, generate the second screen based on the user information, and control the display to display the second screen.

The user information includes at least one of body part information, body analysis information, schedule information, preferred exercise information, or history information.

The instructions, when executed by the at least one processor individually or collectively, cause the electronic apparatus to: determine a target exercise among a plurality of exercises based on the user information, and generate the second screen providing guidance for the target exercise.

The instructions, when executed by the at least one processor individually or collectively, cause the electronic apparatus to: identify a posture of the target exercise based on determining the target exercise, identify a second target part of the authenticated user based on the posture of the target exercise and a touch location of the second target part on the electronic apparatus, generate a second body part UI based on information corresponding to the second target part among the body part information and the touch location, generate the second screen including the second body part UI, and control the display to display the second screen.

The instructions when executed by the at least one processor individually or collectively, cause the electronic apparatus to: identify a cable location corresponding to the target exercise based on a cable location table stored in the memory when a grip is required in the target exercise, and provide a UI indicating the cable location.

According to an aspect of the disclosure, a controlling method of an electronic apparatus, the controlling method includes: acquiring audio data including a voice of a user; performing a first user authentication based on the audio data; based on identifying that the first user authentication is successful, displaying a first screen including a first body part user interface (UI) corresponding to an authenticated user; acquiring touch data including a user touch while the first screen is displayed; performing second user authentication based on the touch data; and based on identifying that the second user authentication is successful, displaying a second screen for exercise content.

The performing the first user authentication includes: acquiring a first difference value between the audio data and registered voice data stored in the electronic apparatus, and identifying that the first user authentication is successful based on the first difference value being less than a first threshold value.

The displaying the first screen further includes: based on identifying the first user authentication is successful, acquiring body part information corresponding to the authenticated user; identifying a preset first target part among a plurality of body parts included in the body part information; generating the first body part UI based on the preset first target part; generating the first screen including the first body part UI; and displaying the first screen.

The controlling method further includes based on the first screen being displayed, activating a sensor for acquiring the touch data.

The performing the second user authentication includes: acquiring a second difference value between the touch data and registered touch data stored in the electronic apparatus; and identifying that the second user authentication is successful based on the second difference value being less than a second threshold value.

According to an aspect of the disclosure, a non-transitory computer readable medium having instructions stored therein, which when executed by a processor in an electronic apparatus cause the electronic apparatus to execute a method including: acquiring audio data including a voice of a user; performing a first user authentication based on the audio data; based on identifying that the first user authentication is successful, displaying a first screen including a first body part user interface (UI) corresponding to an authenticated user; acquiring touch data including a user touch while the first screen is displayed; performing second user authentication based on the touch data; and based on identifying that the second user authentication is successful, displaying a second screen for exercise content.

### BRIEF DESCRIPTION OF THE DRAWINGS

FIG. 1 is a diagram for describing a user exercising using an electronic apparatus according to one or more embodiments.

FIG. 2 is a block diagram illustrating the electronic apparatus according to an embodiment.

FIG. 3 is a block diagram for describing a specific configuration of the electronic apparatus of FIG. 2 according to an embodiment.

FIG. 4 is a diagram for describing a system including the electronic apparatus, a terminal apparatus, and a server according to an embodiment.

FIG. 5 is a diagram for describing an operation of providing a screen related to exercise in the electronic apparatus according to an embodiment.

FIG. 6 is a diagram for describing an operation of performing two authentications according to an embodiment.

FIG. 7 is a diagram for describing a second authentication operation according to an embodiment.

FIG. 8 is a diagram for describing a cable location according to an embodiment.

FIG. 9 is a diagram for describing an operation of acquiring information related to a target exercise using user information according to an embodiment.

FIG. 10 is a diagram for describing information related to a target exercise according to an embodiment.

FIG. 11 is a diagram for describing a UI indicating a cable location according to an embodiment.

FIG. 12 is a diagram for describing the UI indicating the cable location according to an embodiment.

FIG. 13 is a diagram for describing an operation of analyzing a user's body measurement information according to an embodiment.

FIG. 14 is a diagram for describing a distinguished body type according to an embodiment.

FIG. 15 is a diagram for describing guide information according to a body type according to an embodiment.

FIG. 16 is a diagram for describing a structure of the electronic apparatus and the terminal apparatus according to an embodiment.

FIG. 17 is a diagram for describing a grip connected to the electronic apparatus according to an embodiment.

FIG. 18 is a diagram for describing a plurality of sensors included in the grip and the electronic apparatus according to an embodiment.

FIG. 19 is a diagram for describing an operation performed by each component in the electronic apparatus according to an embodiment.

FIG. 20 is a diagram for describing the structure of the electronic apparatus according to an embodiment.

FIG. 21 is a diagram for describing an operation of recognizing a user touch according to an embodiment.

FIG. 22 is a diagram for describing an operation of guiding a user to perform a target exercise according to an embodiment.

FIG. 23 is a diagram for describing an operation of recognizing a user touch to perform secondary authentication according to an embodiment.

FIG. 24 is a diagram for describing an operation of providing a guide UI related to exercise according to an embodiment.

FIG. 25 is a diagram for describing an embodiment of providing a guide screen to a user according to an embodiment.

FIG. 26 is a diagram for describing an embodiment of providing a guide screen to a user according to an embodiment.

FIG. 27 is a diagram for describing an embodiment in which a user requests a guide screen according to an embodiment.

FIG. 28 is a diagram for describing an embodiment in which the user requests the guide screen according to an embodiment.

FIG. 29 is a diagram for describing an embodiment in which a user is replaced according to an embodiment.

FIG. 30 is a diagram for describing an embodiment in which the user is replaced according to an embodiment.

FIG. 31 is a diagram for describing an embodiment in which the user is replaced according to an embodiment.

FIG. 32 is a diagram for describing an embodiment in which the user is replaced according to an embodiment.

FIG. 33 is a diagram for describing an operation of providing a feedback UI according to an embodiment.

FIG. 34 is a diagram for describing an operation of providing the feedback UI according to an embodiment.

FIG. 35 is a diagram for describing an operation of displaying the UI indicating the cable location according to an embodiment.

FIG. 36 is a diagram for describing an operation of providing exercise content according to an embodiment.

FIG. 37 is a diagram for describing an operation of providing guide information related to exercise content according to an embodiment.

FIG. 38 is a diagram for describing a screen related to exercise content according to an embodiment.

FIG. 39 is a diagram for describing a body part UI corresponding to exercise according to an embodiment.

FIG. 40 is a diagram for describing the body part UI corresponding to the exercise according to an embodiment.

FIG. 41 is a diagram for describing the body part UI corresponding to the exercise according to an embodiment.

FIG. 42 is a diagram for describing the body part UI corresponding to the exercise according to an embodiment.

FIG. 43 is a diagram for describing a control operation of the electronic apparatus according to an embodiment.

### DETAILED DESCRIPTION

Hereinafter, certain embodiments of the disclosure will be described in detail with reference to the accompanying drawings.

General terms that are currently widely used were selected as terms used in embodiments of the disclosure in consideration of functions in the disclosure, but may be changed depending on the intention of those skilled in the art or a judicial precedent, the emergence of a new technique, and the like. In addition, in a specific case, terms arbitrarily chosen by an applicant may exist. In this case, the meaning of such terms will be mentioned in detail in a corresponding description portion of the disclosure. Therefore, the terms used in embodiments of the disclosure should be defined on the basis of the meaning of the terms and the contents throughout the disclosure rather than simple names of the terms.

In the disclosure, an expression “have”, “may have”, “include”, “may include”, or the like, indicates existence of a corresponding feature (for example, a numerical value, a function, an operation, a component such as a part, or the like), and does not exclude existence of an additional feature.

An expression “at least one of A or B” is to be understood to represent “A” or “B” or “both A and B.”

Expressions “first”, “second”, or the like, used in the disclosure may indicate various components regardless of a sequence and/or importance of the components, will be used only in order to distinguish one component from the other components, and do not limit the corresponding components.

When it is mentioned that any component (for example, a first component) is (operatively or communicatively) coupled with/to or is connected to another component (for example, a second component), it is to be understood that any component is directly coupled to another component or may be coupled to another component through the other component (for example, a third component).

Singular expressions are intended to include plural expressions unless the context clearly indicates otherwise. It will be further understood that terms “include” or “formed of” used in the present specification specify the presence of features, numerals, steps, operations, components, parts, or combinations thereof mentioned in the present specification, but do not preclude the presence or addition of one or more other features, numerals, steps, operations, components, parts, or combinations thereof.

In the disclosure, a ‘module’ or a ‘unit’ may perform at least one function or operation, and be implemented by hardware or software or be implemented by a combination of hardware and software. In addition, a plurality of “modules” or a plurality of “˜ers/ors” may be integrated in at least one module and be implemented by at least one processor except for a ‘module’ or an ‘˜er/or’ that needs to be implemented by specific hardware.

In the disclosure, the term user may refer to a person using an electronic apparatus or an apparatus (for example, an artificial intelligence electronic apparatus) using an electronic apparatus.

Hereinafter, an embodiment of the disclosure will be described in detail with reference to the accompanying drawings.

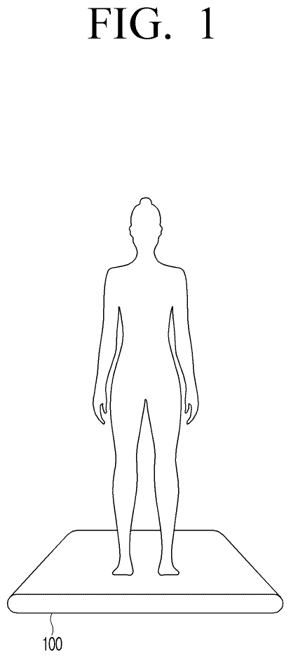

FIG. 1 is a diagram for describing a user exercising using an electronic apparatus according to an embodiment.

Referring to FIG. 1, an electronic apparatus 100 may be an apparatus including a display 140. The electronic apparatus 100 may be an apparatus that provides various types of information related to exercise to a user through the display. The electronic apparatus 100 may be described as an exercise electronic apparatus, an exercise assistance device, an exercise guide device, etc. The exercise electronic apparatus may be incorporated into any type of exercise equipment known to one of ordinary skill in the art such as an exercise bike, treadmill, elliptical, etc.

The electronic apparatus 100 may include a touch sensor for sensing a user touch. The electronic apparatus 100 may receive a user touch through the touch sensor and perform various operations based on the received user touch.

In one or more examples, the electronic apparatus 100 may perform a user authentication operation based on the user touch. The electronic apparatus 100 may acquire touch data related to a user's body (e.g., hands, feet, etc.) based on the user touch. The electronic apparatus 100 may identify whether the user is an authenticated user using the touch data related to the user's body. For example, the user may be authenticated based on a fingerprint or handprint. Furthermore, the user may be authenticated based on an image of the user, where facial recognition or eye recognition is performed.

After the user is authenticated, the electronic apparatus 100 may perform an exercise guide operation based on the user touch. The electronic apparatus 100 may identify whether a user's exercise posture is appropriate based on the user touch. When the user's exercise posture is not appropriate, the electronic apparatus 100 may provide a user with a guide for a corrected exercise posture.

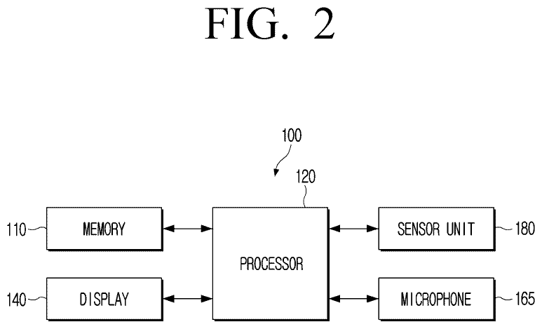

FIG. 2 is a block diagram illustrating the electronic apparatus according to an embodiment.

The electronic apparatus 100 may include a memory 110 storing instructions, a microphone 165, a sensor unit 180, a display 140, and at least one processor 120 including processing circuitry.

The at least one processor 120 may provide exercise content to a user. The at least one processor 120 may specify a user before providing the exercise content.

For example, at least one processor 120 may perform authentication for a representative user who has already been registered in the electronic apparatus 100. At least one processor 120 may request a user's voice according to a preset event. When the user's voice is received, at least one processor 120 may identify whether the received user's voice is identical to a voice of a previously registered representative user. The previously registered representative user may correspond to a voice of the user recorded, for example, during an authentication set up procedure.

In one or more examples, at least one processor 120 may perform authentication for an arbitrary user. At least one processor 120 may provide a UI for guiding a user to utter the user's voice for first user authentication (or a Primary user authentication). When the user's voice is received through the provided UI, at least one processor 120 may identify whether the received user's voice is a previously registered voice.

At least one processor 120 may acquire audio data including the user's voice through the microphone 165. At least one processor 120 may perform the first user authentication based on the audio data.

At least one processor 120 may acquire a first difference value between the audio data and the registered voice data stored in the memory 110. When the first difference value is less than a first threshold value, at least one processor 120 may identify that the first user authentication is successful.

When the first user authentication is successful, at least one processor 120 may control the display 140 to display a first screen including a first body part UI corresponding to the authenticated user.

When it is identified that the first user authentication is successful, at least one processor 120 may obtain body part information corresponding to the authenticated user.

The body part information may be information representing a part of a user's body. The body part information may include information representing a shape and size of a part of a body of the authenticated user.

At least one processor 120 may identify a preset first target part among a plurality of body parts included in the body part information. The first target part may represent a part that is touched by the electronic apparatus 100 to perform second(or secondary) user authentication.

For example, the first target part may be a left sole and a right sole.

For example, the first target part may be the left sole.

For example, the first target part may be the right sole.

The first target part may be pre-stored in the memory 110. The first target part may be changed according to a user's settings indicating a desired preference.

At least one processor 120 may generate a first body part UI based on the first target part. At least one processor 120 may extract first target data corresponding to the first target part from among body data of the authenticated user included in the body part information. At least one processor 120 may generate the first body part UI based on the first target data.

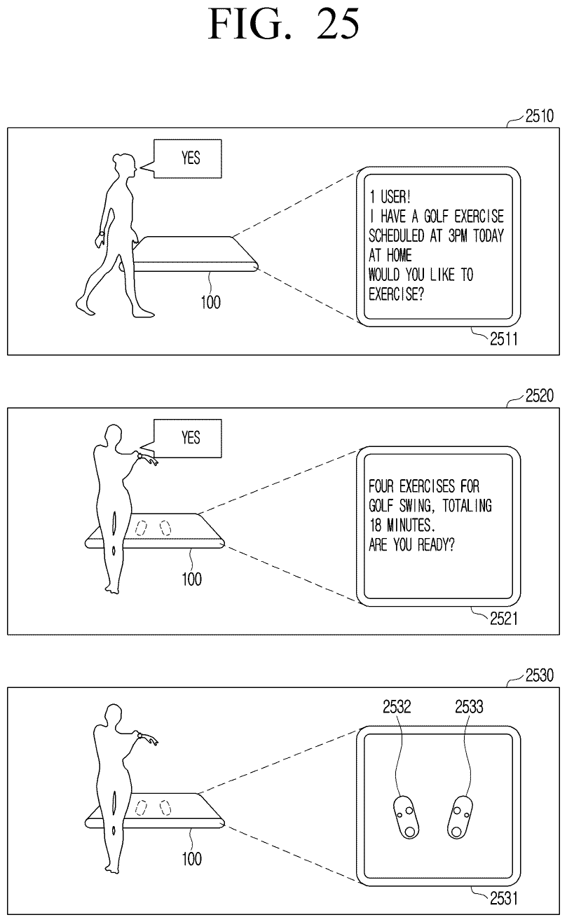

At least one processor 120 may generate the first screen including the first body part UI. At least one processor 120 may control the display 140 to display the first screen. A description related to the first body part UI and the first screen is described in an embodiment 2530 of FIG. 25.

When the first screen is displayed, at least one processor 120 may activate the sensor unit 180. While the sensor unit 180 is in an off state or a sleep mode, at least one processor 120 may transmit an activation notification to the sensor unit 180 according to a preset event (display of the first screen). According to the activation notification, the sensor unit 180 may be changed from an off state to an on state. When the sensor unit 180 is changed to the on state according to the activation notification, the sensor unit 180 may acquire sensing data (or touch data) by a user touch. At least one processor 120 may acquire the touch data including the user touch through the sensor unit 180 while the first screen is displayed. Although the sensor is described as being in an off state or an on state, the sensor may also have a sleep state or power saving state in which the sensor unit 180 is not completely powered off.

At least one processor 120 may perform the second user authentication based on the touch data. The second user authentication may be performed through a touch action rather than a voice.

At least one processor 120 may acquire a second difference value between the touch data and the registered touch data stored in the memory 110. When the difference value is less than a second threshold value, at least one processor 120 may identify that the second user authentication is successful.

When the second user authentication is successful, at least one processor 120 may control the display 140 to display a second screen for exercise content.

When it is identified that the second user authentication is successful, at least one processor 120 may acquire user information corresponding to the authenticated user. At least one processor 120 may generate the second screen based on the user information and control the display 140 to display the second screen.

The user information may include at least one of the body part information, the body analysis information, schedule information, the preferred exercise information, or the history information.

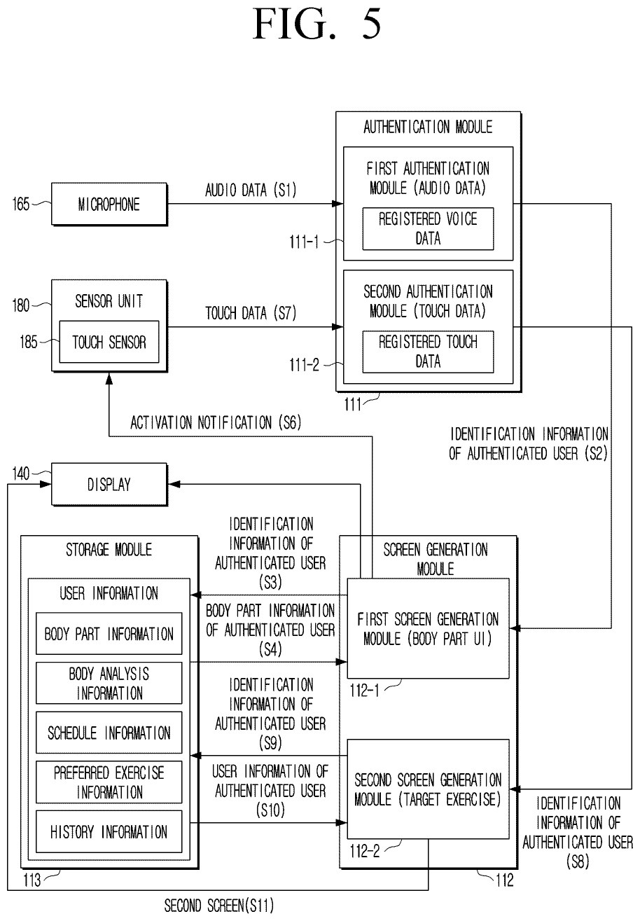

Specific details related to the user information are described in FIGS. 5, 9, 13, and 14.

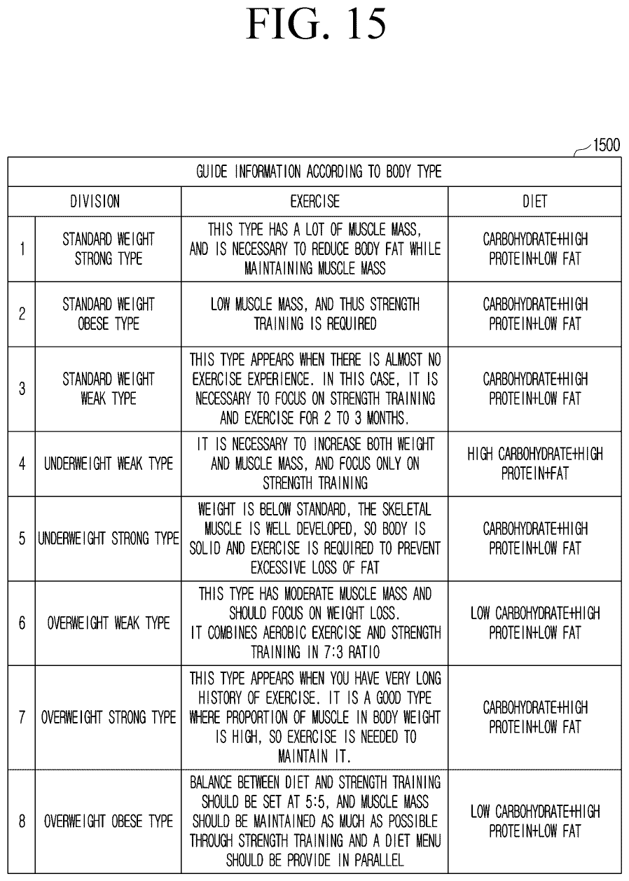

At least one processor 120 may determine a target exercise among a plurality of exercises based on the user information and generate the second screen for guiding the target exercise. The target exercise may be an exercise recommended to a user. At least one processor 120 may determine the target exercise to recommend to the user based on the user information. The target exercise may be an exercise that the user currently needs the most. The operation of determining the target exercise by the body type is described in FIG. 15.

For example, when a user selects a specific exercise, at least one processor 120 may determine the selected exercise as the target exercise.

When the target exercise is determined, at least one processor 120 may identify the posture of the target exercise. At least one processor 120 may identify the second target part of the authenticated user who should touch the electronic apparatus based on the posture of the target exercise and a touch location of the second target part.

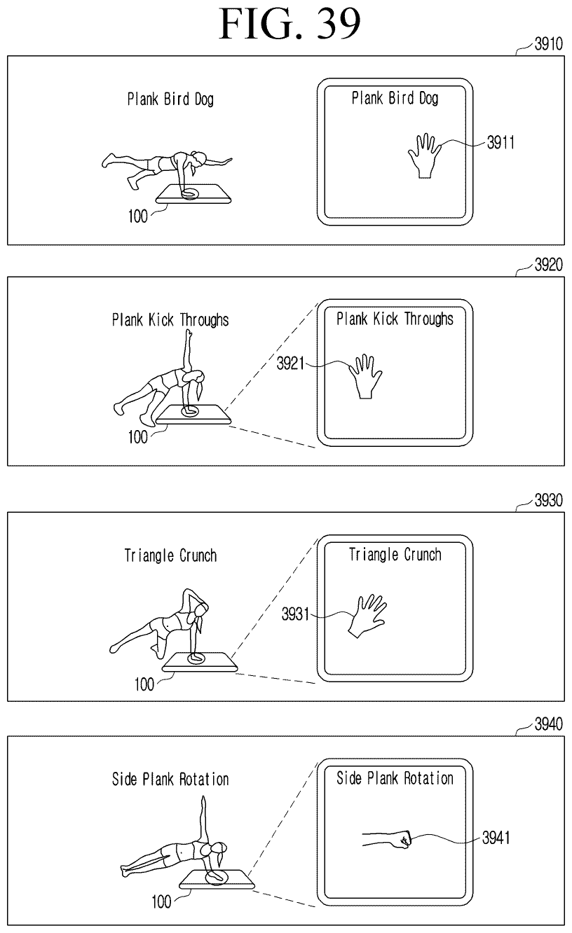

The second target part may be a part that is touched with the electronic apparatus 100 for the target exercise. The second target part may vary depending on the target exercise. A description related thereto is described in FIGS. 39 to 42.

At least one processor 120 may generate a second body part UI based on information corresponding to the second target part among the body part information and the touch location. At least one processor 120 may generate the second screen including the second body part UI and control the display 140 to display the second screen.

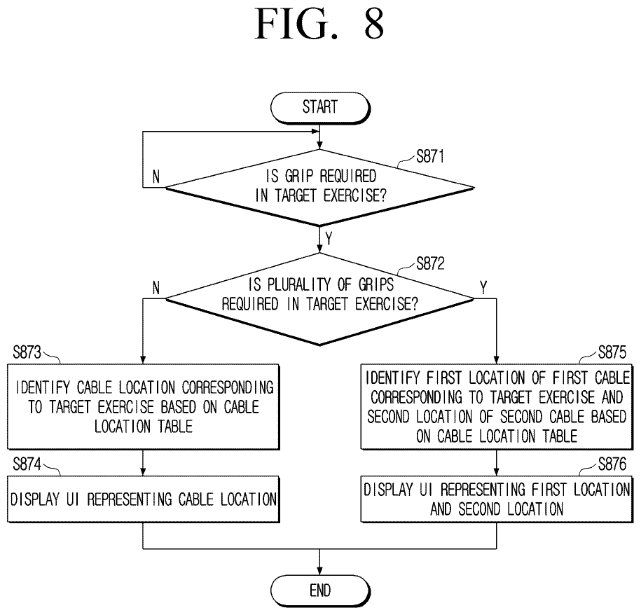

When a grip is required in the target exercise, at least one processor 120 may identify a cable location corresponding to the target exercise based on a cable location table stored in the memory 110 and provide a UI indicating the cable location. Descriptions related thereto are described in FIGS. 8, 10, 11, 12, 17, and 35.

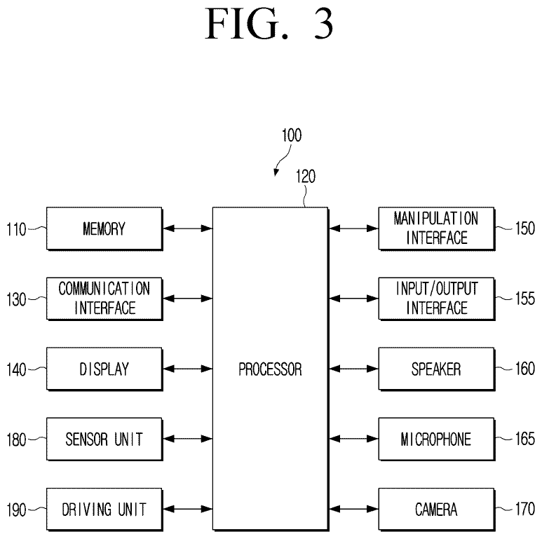

FIG. 3 is a block diagram for describing a specific configuration of the electronic apparatus of FIG. 2 according to an embodiment.

Referring to FIG. 3, the electronic apparatus 100 may include at least one of the memory 110, at least one processor 120, a communication interface 130, a display 140, a manipulation interface 150, an input/output interface 155, a speaker 160, a microphone 165, or a camera 170.

The memory 110 may be implemented by an internal memory such as a read-only memory (ROM) (for example, an electrically erasable programmable read-only memory (EEPROM)), a random access memory (RAM), or the like, included in the at least one processor 120 or be implemented by a memory separate from at least one processor 120. The memory 110 may be implemented in a form of a memory embedded in the electronic apparatus 100 or a form of a memory attachable to and detachable from the electronic apparatus 100, depending on a data storage purpose. For example, data for driving the electronic apparatus 100 may be stored in the memory embedded in the electronic apparatus 100, and data for an extension function of the electronic apparatus 100 may be stored in the memory attachable to and detachable from the electronic apparatus 100.

The memory embedded in the electronic apparatus 100 may be implemented by at least one of a volatile memory (for example, a dynamic RAM (DRAM), a static RAM (SRAM), a synchronous dynamic RAM (SDRAM), or the like) or a non-volatile memory (for example, a one time programmable ROM (OTPROM), a programmable ROM (PROM), an erasable and programmable ROM (EPROM), an electrically erasable and programmable ROM (EEPROM), a mask ROM, a flash ROM, a flash memory (for example, a NAND flash, a NOR flash, or the like), a hard drive, or a solid state drive (SSD)), and the memory attachable to and detachable from the electronic apparatus 100 may be implemented in a form such as a memory card (for example, a compact flash (CF), a secure digital (SD), a micro-SD, a mini-SD, an extreme digital (xD), a multi-media card (MMC), or the like), an external memory (for example, a universal serial bus (USB) memory) connectable to a USB port, or the like.

The memory 110 may store at least one instruction. At least one processor 120 may perform various operations based on an instruction stored in the memory 110.

At least one processor 120 may be implemented by a digital signal processor (DSP), a microprocessor, or a time controller (TCON) that processes a digital signal. However, the at least one processor 120 is not limited thereto, but may include one or more of a central processing unit (CPU), a micro controller unit (MCU), a micro processing unit (MPU), a controller, an application processor (AP), a graphics-processing unit (GPU), a communication processor (CP), and an advanced reduced instruction set computer (RISC) machines (ARM) processor, or may be defined by these terms. At least one processor 120 may be implemented by a system-on-chip (SoC) or a large scale integration (LSI) in which a processing algorithm is embedded, or may be implemented in a field programmable gate array (FPGA) form. At least one processor 120 may perform various functions by executing computer executable instructions stored in the memory.

The communication interface 130 is a component performing communication with various types of external apparatuses depending on various types of communication manners. The communication interface 130 may include a wireless communication module or a wired communication module. Each communication module may be implemented in the form of at least one hardware chip.

The wireless communication module may be a module that communicates with an external device wirelessly. For example, the wireless communication module may include at least one of a WiFi module, a Bluetooth module, an infrared communication module, or other communication modules.

The WiFi module and the Bluetooth module may perform communication in a WiFi manner and a Bluetooth manner, respectively. In the case of using the WiFi module or the Bluetooth module, various connection information such as a service set identifier (SSID), a session key, and the like, is first transmitted and received, communication is connected using the connection information, and various information may then be transmitted and received.

The infrared communication module performs communication according to an infrared data association (IrDA) technology of wirelessly transmitting data to a short distance using an infrared ray positioned between a visible ray and a millimeter wave.

Other wireless communication modules may include at least one communication chip performing communication according to various wireless communication standards such as zigbee, 3rd generation (3G), 3rd generation partnership project (3GPP), long term evolution (LTE), LTE advanced (LTE-A), 4th generation (4G), 5th generation (5G), and the like, in addition to the communication manner described above.

The wired communication module may be a module that communicates with an external device in a wired manner. For example, the wired communication module may include at least one of a local area network (LAN) module, an Ethernet module, a pair cable, a coaxial cable, a fiber optic cable, or an ultra wide-band (UWB) module.

According to an embodiment, the communication interface 130 may use the same communication module (for example, the WiFi module) to communicate with an external device such as a remote control and an external server.

According to an embodiment, the communication interface 130 may use different communication modules to communicate with an external device such as a remote control and an external server. For example, the communication interface 130 may use at least one of the Ethernet module or the WiFi module to communicate with the external server, and may use a Bluetooth module to communicate with the external device such as the remote control. However, this is only an example, and the communication interface 130 may use at least one of various communication modules in a case in which it communicates with a plurality of external devices or external servers.

The display 140 may be implemented by various types of displays such as a liquid crystal display (LCD), an organic light emitting diode (OLED) display, a plasma display panel (PDP), and the like. A driving circuit, a backlight unit, and the like, that may be implemented in a form such as an amorphous silicon thin film transistor (a-si TFT), a low temperature poly silicon (LTPS) TFT, and an organic TFT (OTFT), may be included in the display 140. The display 140 may be implemented as a touch screen combined with a touch sensor, a flexible display, a three-dimensional display (3D display), etc. According to an embodiment of the disclosure, the display 140 may include not only a display panel that outputs an image, but also a bezel that houses a display panel. In particular, according to an embodiment of the disclosure, the bezel may include a touch sensor for detecting user interaction.

The manipulation interface 150 may be implemented as a device such as a button, a touch pad, a mouse, and a keyboard, or may be implemented as a touch screen capable of performing the above-described display function and manipulation input function together. In one or more examples, the button may be various types of buttons such as a mechanical button, a touch pad, a wheel, and the like, formed in any region such as a front surface portion, a side surface portion, a rear surface portion, and the like, of a body appearance of the electronic apparatus 100.

The input/output interface 155 may be an interface of any one of a high definition multimedia interface (HDMI), a mobile high-definition link (MHL), a universal serial bus (USB), a display port (DP), Thunderbolt, a video graphics array (VGA) port, an RGB port, a D-subminiature (D-SUB), and a digital visual interface (DVI). The input/output interface 155 may input/output at least one of audio signal or the video signal. Depending on the implementation example, the input/output interface 155 may include a port for inputting/outputting only an audio signal and a port for inputting/outputting only a video signal as separate ports, or may be implemented as a single port for inputting/outputting both an audio signal and a video signal. The electronic apparatus 100 may transmit at least one of the audio signal or the video signal to an external device (e.g., an external display device or an external speaker) through the input/output interface 155. An output port included in the input/output interface 155 may be connected to an external device, and the electronic apparatus 100 may transmit at least one of the audio signal or the video signal to the external device through the output port.

The input/output interface 155 may be connected to a communication interface. The input/output interface 155 may transmit information received from an external device to the communication interface, or transmit information received through the communication interface to the external device.

The speaker 160 may be a component that outputs not only various audio data but also various notification sounds, voice messages, etc.

The microphone 165 is a configuration for receiving a user's voice or other sounds and converting the user's voice or other sounds into audio data. The microphone 165 may receive the user's voice in an activated state. For example, the microphone 165 may be formed integrally on an upper side, front direction, a side direction, etc., of the electronic apparatus 100. The microphone 165 may include various components such as a microphone for collecting the user's voice in an analog form, an amplifier circuit for amplifying the collected user's voice, an A/D conversion circuit for sampling the amplified user's voice and converting the sampled user's voice into a digital signal, and a filter circuit for removing noise components from the converted digital signal.

The camera 170 is configured to generate a captured image by capturing a subject, in which the captured image is a concept including both a moving image and a still image. The camera 170 may acquire an image of at least one external device, and may be implemented as a camera, a lens, an infrared sensor, or the like.

The camera 170 may include a lens and an image sensor. A type of lens includes a general general-purpose lens, a wide-angle lens, a zoom lens, and the like, and may be determined according to the type, characteristic, use environment, and the like of the electronic apparatus 100. As the image sensor, a complementary metal oxide semiconductor (CMOS), a charge coupled device (CCD), etc., may be used.

The sensor unit 180 may include at least one sensor connected to the electronic apparatus 100. The sensor unit 180 may include at least one of a gyroscope sensor, a horizontal sensor, an infrared sensor, a grip force sensor, a vibration sensor, a body composition sensor, a weight sensor, a pressure sensor, a touch sensor, a speed sensor, an acceleration sensor, a motion sensor, or a tension sensor. The sensor unit 180 may include two or more sensors. The sensor unit 180 may be referred to as a sensor or sensor circuitry. The arrangement positions of the two or more sensors may be different.

The sensor unit 180 is a word encompassing at least one sensor, and may not necessarily mean one hardware module. For example, a first sensor may be arranged in a first module and a second sensor may be arranged in a second module. The first module and the second module may be physically separate hardware components.

The driving unit 190 may transmit physical force to components included in the electronic apparatus 100. The driving unit 190 may include a motor. The driving unit 190 may supply power to the motor to generate physical force and transmit the generated physical force to hardware components included in the electronic apparatus 100.

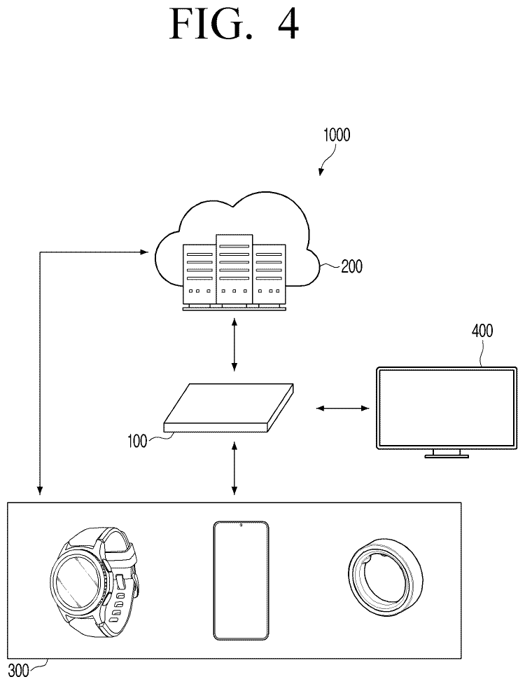

FIG. 4 is a diagram for describing a system including the electronic apparatus, a terminal apparatus, and a server according to an embodiment.

Referring to FIG. 4, the electronic apparatus 100 may communicate with at least one of the server 200 or the terminal apparatus 300.

For example, the electronic apparatus 100 may communicate with the server 200.

For example, the electronic apparatus 100 may communicate with the terminal apparatus 300.

For example, the electronic apparatus 100 may be connected to both the server 200 and the terminal apparatus 300.

The server 200 may be a server that provides the exercise content. The server 200 may be a server that stores the exercise content provided by the electronic apparatus 100. The electronic apparatus 100 may request the exercise content from the server 200. The server 200 may transmit the exercise content to the electronic apparatus 100 in response to the request of the electronic apparatus 100. The server 200 may periodically update the electronic apparatus with new or modified exercise content.

For example, the exercise content may be content related to a user authenticated in the electronic apparatus 100.

For example, the exercise content may be the content input (or selected) by the user in the electronic apparatus 100. The electronic apparatus 100 may receive a user input requesting to provide specific exercise content.

The terminal apparatus 300 may be a device carried by a user. The terminal apparatus 300 may include a smart watch, a smart phone, a smart ring, etc. The terminal apparatus 300 may acquire user body analysis information or receive user input. The terminal apparatus 300 may transmit the acquired user body analysis information or user input to the electronic apparatus 100.

For example, the terminal apparatus 300 may manage a user's exercise data through an application stored in the terminal apparatus 300. The exercise data may include at least one of an exercise schedule, an exercise list, an exercise history, exercise performance, or an exercise method. The terminal apparatus 300 may transmit the exercise data to the server 200. The server 200 may provide the terminal apparatus 300 with an exercise function, an exercise guide, exercise content, and exercise performance information related to the electronic apparatus 100. The exercise function may include a type of exercises that can be provided by the electronic apparatus 100. The terminal apparatus 300 may receive various types of information related to the electronic apparatus 100 from the server 200.

For example, the terminal apparatus 300 may acquire measurement data. The measurement data may include at least one of bio-information, heart rate, electrocardiogram, blood pressure, respiration, body temperature, pulse oxygen saturation, or calories consumed. The terminal apparatus 300 may transmit the measurement data to the electronic apparatus 100. The electronic apparatus 100 may analyze the measurement data received from the terminal apparatus 300. The measurement data may be obtained by one or more biometric sensors configured to measure biometric information such as heart rate, electrocardiogram, blood pressure, respiration, body temperature, pulse oxygen saturation, and calories consumed.

The terminal apparatus 300 may be implemented as a plurality of devices. The plurality of devices may be linked with each other.

The electronic apparatus 100 may acquire the measurement data. The measurement data may include at least one of body composition information, weight information, pressure information, or balance information. The electronic apparatus 100 may acquire exercise data. The exercise data may include at least one of an exercise type, an exercise set, the number of repetitions of exercise, whether exercise is repeated, weight, or exercise duration. The electronic apparatus 100 may transmit the measurement data and the exercise data to the server 200. The server 200 may analyze the measurement data and the exercise data transmitted from the electronic apparatus 100 to determine the target exercise. The server 200 may generate information related to the target exercise. The information related to the target exercise may include at least one of the guide information, the program information, or the exercise performance information. The server 200 may transmit the information related to the target exercise to the electronic apparatus 100.

For example, the terminal apparatus 300 may be connected to an external apparatus 400. The external apparatus 400 may include a camera and a display. For example, the electronic apparatus 100 may be implemented as a TV, a mobile service robot, a robot vacuum cleaner, a smart camera, etc.

The external apparatus 400 may capture a user using the camera. The external apparatus 400 may acquire captured data including the user. The external apparatus 400 may transmit the captured data to the server 200. The external apparatus 400 may display the exercise content. The external apparatus 400 may store history information indicating whether to provide the exercise content. The external apparatus 400 may transmit the history information to the server 200.

The server 200 may provide the terminal apparatus 400 with the exercise function, the exercise guide, the exercise content, and the exercise performance information related to the electronic apparatus 100. The exercise function may include the type of exercises that can be provided by the electronic apparatus 100. The external apparatus 400 may display the information received from the server 200. For example, the external apparatus 400 may receive the exercise content from the server 200 and display the received exercise content.

The electronic apparatus 100 may communicate with the server 200 or the terminal apparatus 300 through various communication methods.

For example, the electronic apparatus 100 may be connected to the server 200 via a first communication method (e.g., WiFi, wired Internet).

For example, the electronic apparatus 100 may be connected to the terminal apparatus 300 via a second communication method (e.g., Bluetooth).

FIG. 5 is a diagram for describing an operation of providing a screen related to exercise in the electronic apparatus according to an embodiment.

Referring to FIG. 5, the electronic apparatus 100 may include at least one of an authentication module 111, a screen generation module 112, a storage module 113, a display 140, a microphone 165, or a sensor unit 180. For example, the authentication module 111, the screen generation module 112, and the storage module 113 may be included in the memory 110. Each module illustrated in FIG. 5 may be implemented by one or more processors.

The authentication module 111 may include at least one of a first authentication module 111-1 or a second authentication module 111-2.

The screen generation module 112 may include at least one of a first screen generation module 112-1 or a second screen generation module 112-2.

The electronic apparatus 100 may receive audio data through the microphone 165. The electronic apparatus 100 may transmit the received audio data to the first authentication module 111-1 (S1).

The first authentication module 111-1 may receive the audio data from the microphone 165. The first authentication module 111-1 may be a module that performs user authentication using voice.

The first authentication module 111-1 may perform the first user authentication based on the audio data. The first authentication module 111-1 may identify whether the received audio data matches pre-stored registered voice data. The first authentication module 111-1 may store the registered voice data including user-specific voice data. The first authentication module 111-1 may perform the first user authentication by comparing the received audio data with the stored registered voice data. The first user authentication may be described as the voice authentication.

When the first user authentication is successful (or completed) based on the audio data (e.g., user is recognized based on voice data), the first authentication module 111-1 may acquire identification information of the authenticated user. The first authentication module 111-1 may transmit the identification information of the authenticated user to the first screen generation module 112-1 (S2).

The first screen generation module 112-1 may be a module that generates a screen for guiding a user touch input. The first screen generation module 112-1 may be a module that generates a screen for guiding user's second user authentication.

The first screen generation module 112-1 may acquire the identification information of the authenticated user from the first authentication module 111-1. The first screen generation module 112-1 may transmit the identification information of the authenticated user to the storage module 113 (S3). The first screen generation module 112-1 may request the body part information corresponding to the identification information of the authenticated user from the storage module 113.

The first screen generation module 112-1 may transmit a first control signal for requesting the body part information of the authenticated user. The first control signal may include the identification information of the authenticated user. The first screen generation module 112-1 may transmit the first control signal to the storage module 113.

The storage module 113 may store the body part information. The body part information may include information representing a body part of a user. The body part information may include information representing each body part of the authenticated user. The body part information may include information representing a shape and size of a part of the body of the authenticated user. The size and shape of the body part may be different for each user. For example, the body part may include at least one of a hand, a finger, or a foot.

The storage module 113 may store the body part information for at least one user. When there are multiple previously registered users, the storage module 113 may include the body part information for each of the multiple users. The storage module 113 may provide the body part information corresponding to the user requested by the first screen generation module 112-1.

The body part information may include information on at least one body part of the user. For example, the body part information may include at least one of palms, fingers, soles, toes, head, neck, back, waist, hips, thighs, stomach, arms, knees, legs, or shoulders. The body part information may include all information on the body part that comes into contact with the electronic apparatus 100.

The body part may be distinguished into right and left. For example, the body part information may include at least one of left palm, right palm, left finger, right finger, left sole, right sole, left toe, or right toe. However, the embodiments are not limited to this configuration. For example, the body part may be distinguished between upper and lower body parts (e.g., distinguishing between upper torso and lower torso).

The storage module 113 may receive the first control signal from the first screen generation module 112-1. The storage module 113 may identify the body part information based on the identification information of the authenticated user included in the first control signal. The identified body part information may be the body part information of the authenticated user, and the body part information corresponding to the identification information of the authenticated user. The storage module 113 may transmit the body part information of the authenticated user to the first screen generation module 112-1 (S4).

The first screen generation module 112-1 may receive the body part information of the authenticated user from the storage module 113. The first screen generation module 112-1 may acquire the body part information of the authenticated user based on the identification information of the authenticated user. The first screen generation module 112-1 may generate the first screen based on the body part information of the authenticated user.

The first screen generation module 112-1 may generate the first screen including the body part UI of the authenticated user. The first screen may be a screen for guiding the user's first user authentication. The first screen may be a screen for guiding the user to input the user touch through the body part UI included in the first screen. The body part UI included in the first screen may be different for each user. The first screen generation module 112-1 may transmit the first screen to the display 140 (S5).

The body part UI may be a UI representing a part of the body of the authenticated user. The body part UI may represent the same size and shape as a part of the body of the authenticated user. Since the shape and size of the body may be different for each user, the body part UI may also be different for each user.

The body part information may represent multiple body parts of the user. The body part UI may correspond to a target part. Since the first screen is for the second user authentication, the body part UI may not need to represent all body parts. The target part may be a preset part. The preset part may be a part corresponding to the registered touch data stored in the second authentication module 111-2. The target part may be a preset target part. The target part may be different for different users.

The operation of the first screen generation module 112-1 to determine the target part may vary.

For example, the first screen generation module 112-1 may request only the body part information (partial data) corresponding to the target part from the storage module 113. The storage module 113 may transmit only the body part information (partial data) corresponding to the target part to the first screen generation module 112-1. The first screen generation module 112-1 may generate the body part UI using only the body part information (partial data) corresponding to the target part. The first screen generation module 112-1 may generate the first screen including the body part UI.

For example, the first screen generation module 112-1 may request the body part information (entire data) representing multiple body parts to the storage module 113. The storage module 113 may transmit the body part information (entire data) representing multiple body parts to the first screen generation module 112-1. The first screen generation module 112-1 may identify the target part among multiple body parts. The first screen generation module 112-1 may acquire the body part information (partial data) corresponding to the target part among the body part information (entire data) representing multiple body parts. The first screen generation module 112-1 may generate the body part UI using only the body part information (partial data) corresponding to the target part. The first screen generation module 112-1 may generate the first screen including the body part UI.

The display 140 may receive the first screen from the first screen generation module 112-1. The display 140 may display the first screen.

The first screen generation module 112-1 may transmit an activation notification to the sensor unit 180 to activate the touch sensor 185 (S6). The sensor unit 180 may activate the touch sensor 185 based on the activation notification. When the activation notification is received, the sensor unit 180 may supply power to the touch sensor 185.

While the first screen is displayed on the display 140, the sensor unit 180 may acquire the touch data. The sensor unit 180 may include a touch sensor 185. The touch sensor 185 may be arranged to correspond to a light output direction of the display 140. The touch sensor 185 may acquire data on the user touch that is input by the user. The touch sensor 185 may acquire an area for the user input touching the display 140 as the touch data. The touch sensor 185 may be arranged parallel to the arrangement direction of the display 140. The touch sensor 185 may transmit the touch data to the second authentication module 111-2 (S7).

For example, the touch sensor 185 may acquire the touch data using a capacitive method. The capacitive method may be a method of measuring a change in capacitance that changes due to the user touch. The touch sensor 185 may measure a distance from the touch sensor 185 to a part of the user's body adjacent to the electronic apparatus 100 using the capacitive method. The touch sensor 185 may generate the touch data representing the capacitance that changes due to the touch.

The touch data may include information on the location where the user touch is recognized. The touch data may be used to determine the shape of the touch data that is in contact.

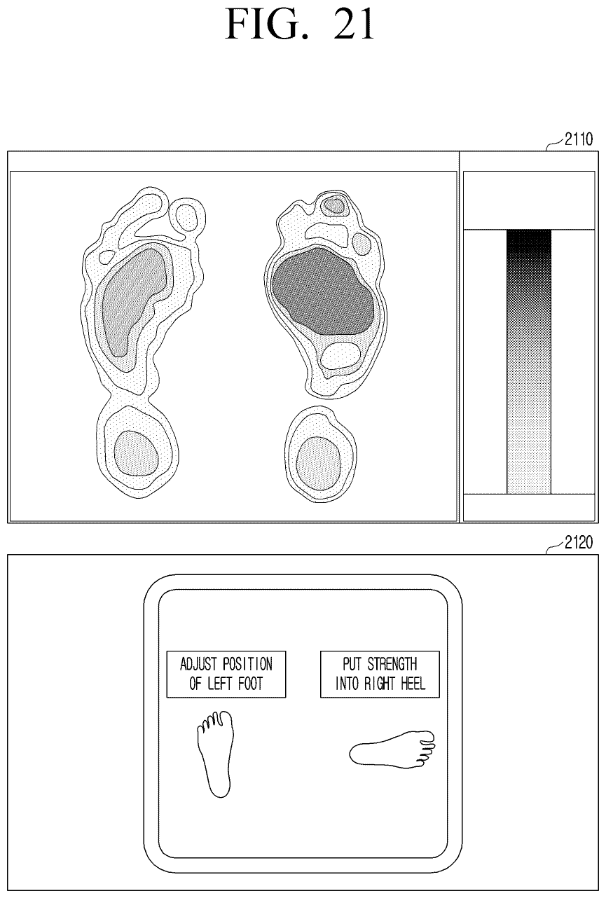

According to an embodiment, the touch data may be used for a user authentication operation together with pressure data. The electronic apparatus 100 may map and store the touch data representing the location of the user touch and pressure data representing the pressure distribution. The electronic apparatus 100 may perform a user authentication function using the touch data and the pressure data. A description related thereto will be made later with reference to FIG. 21.

According to another embodiment, the touch data may be used to determine a hand print, a finger print, a foot print, a toe print, etc. While a dictionary definition of a fingerprint may mean a finger, the fingerprint described in the disclosure may indicate a “print” indicating a pattern, a design, a mark, a trace, etc. As an example, the fingerprint may be described as the pattern. The touch data may include at least one of a hand pattern, a finger pattern, a foot pattern, or a toe pattern.

The second authentication module 111-2 may receive the touch data from the touch sensor 185. The second authentication module 111-2 may be a module that performs the second user authentication based on the touch data. The second user authentication may include an operation of performing the authentication operation based on the touch data. The second authentication module 111-2 may store the registered touch data. The registered touch data may be the touch data that has been pre-stored by the user. The second authentication module 111-2 may acquire the registered touch data through a process of registering the information related to the user.

The second authentication module 111-2 may perform an authentication operation by comparing the touch data received from the touch sensor 185 with the registered touch data. When the received touch data is included in the registered touch data, the second authentication module 111-2 may identify that the authentication is completed (or successful). When the authentication is successful, the second authentication module 111-2 may transmit the identification information of the authenticated user to the second screen generation module 112-2 (S8).

The second screen generation module 112-2 may receive the identification information of the authenticated user from the second authentication module 111-2. The second screen generation module 112-2 may be a module that generates the second screen to guide the target exercise. The second screen generation module 112-2 may be a module that generates various guide screens related to exercise.

When the identification information of the authenticated user is received from the second authentication module 111-2, the second screen generation module 112-2 may transmit the identification information of the authenticated user to the storage module 113 (S9). The second screen generation module 112-2 may request the user information corresponding to the identification information of the authenticated user from the storage module 113.

The second screen generation module 112-2 may generate a second control signal for requesting the user information of the authenticated user. The second control signal may include a signal for requesting user information of a specific user from the storage module 113. The second control signal may include the identification information of the authenticated user.

The user information may include various types of information related to the user required for using the electronic apparatus 100.

For example, the user information may include at least one of the body part information, the body analysis information, the schedule information, the preferred exercise information, or the history information. The body part information may be information indicating the body part of the user. The body analysis information may be information indicating the user's physical ability. The schedule information may include information on the schedule entered by the user. The preferred exercise information may indicate the user's preferred exercise. The history information may include information related to exercise performed by the user in the past. The body part information and the body analysis information may be grouped into body information.

The storage module 113 may store a user information table that stores user information by distinguishing the user information for each user.

The storage module 113 may receive the second control signal from the second screen generation module 112-2. The storage module 113 may receive identification information of an authenticated user included in the second control signal. The storage module 113 may identify user information corresponding to the identification information of the authenticated user. The storage module 113 may identify the user information of the authenticated user in response to the second control signal. The storage module 113 may transmit the user information of the authenticated user to the second screen generation module 112-2 (S10).

The second screen generation module 112-2 may receive the user information of the authenticated user from the storage module 113. The second screen generation module 112-2 may generate the second screen based on the user information of the authenticated user. The second screen may be a guide screen related to exercise.

The second screen generation module 112-2 may determine the target exercise among a plurality of exercises based on the user information of the authenticated user. The target exercise may indicate an exercise to be provided to the user. The second screen generation module 112-2 may determine the target exercise to recommend to the user based on the user information of the authenticated user.

As an example, the second screen generation module 112-2 may acquire body analysis information included in the user information of the authenticated user. The second screen generation module 112-2 may acquire skeletal muscle information included in the body analysis information. The second screen generation module 112-2 may determine that the authenticated user needs strength training based on the skeletal muscle information. The second screen generation module 112-2 may determine the target exercise as the strength training.

For example, the second screen generation module 112-2 may acquire schedule information included in the user information of the authenticated user. The second screen generation module 112-2 may identify that the authenticated user has a golf appointment tomorrow from the schedule information. When the golf appointment is identified, the second screen generation module 112-2 may determine an exercise (e.g., muscle strengthening exercise such as waist exercise, shoulder exercise, arm exercise, wrist exercise, thigh exercise, and hip exercise) that is helpful for golf as the target exercise.

For example, the second screen generation module 112-2 may acquire preferred exercise information included in the user information of the authenticated user. The second screen generation module 112-2 may identify that the user prefers a yoga exercise from the preferred exercise information. The second screen generation module 112-2 may determine the yoga exercise as the target exercise.

For example, the second screen generation module 112-2 may acquire the history information included in the user information of the authenticated user. The second screen generation module 112-2 may identify a recently performed exercise (e.g., yoga exercise) based on the history information. The second screen generation module 112-2 may identify whether the recently performed exercise has been completed. When it is determined that the recently performed exercise has not been completed based on the history information, the second screen generation module 112-2 may determine the incomplete exercise as the target exercise.

When the target exercise is determined, the second screen generation module 112-2 may generate the second screen to guide the target exercise. The second screen generation module 112-2 may transmit the second screen to the display 140 (S11).

The second screen may include various types of information related to the target exercise. The second screen may include guide information for the target exercise. The guide information may include at least one of information indicating the target exercise or information for guiding a posture for the target exercise. The guide information may include at least one of name, time, weight, order, or body part location of the target exercise. The body part location may indicate a touch location of a user who should touch the electronic apparatus 100 when performing the target exercise. The body part location may indicate a location where a part of the user's body should be in contact with the electronic apparatus 100.

For example, the second screen generation module 112-2 may acquire the body part information included in the user information of the authenticated user. The second screen generation module 112-2 may acquire posture information corresponding to the target exercise based on the body part information. The second screen generation module 112-2 may generate the body part UI indicating the touch location of the user based on the posture information corresponding to the target exercise. The second screen generation module 112-2 may generate the second screen including the second body part UI.

For convenience of distinction, the body part UI generated by the first screen generation module 112-1 may be described as the first body part UI. The body part UI generated by the second screen generation module 112-2 may be described as the second body part UI.

The display 140 may receive the second screen from the second screen generation module 112-2. The display 140 may display the second screen. The user may be provided with information on the target exercise through the second screen. The user may easily follow the target exercise through the second screen.

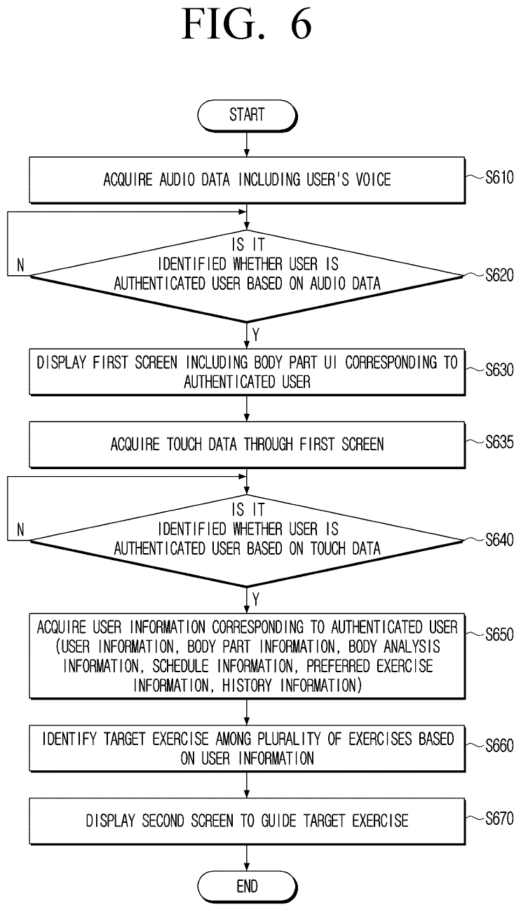

FIG. 6 is a diagram of a flowchart for describing an operation of performing two authentications according to an embodiment.

Referring to FIG. 6, the electronic apparatus 100 may acquire the audio data including the user's voice (S610). The electronic apparatus 100 may perform the first user authentication based on the audio data. The first user authentication may be voice authentication.

The electronic apparatus 100 may identify whether the user is the authenticated user based on the audio data (S620). The electronic apparatus 100 may perform the first user authentication by comparing the received audio data with the pre-stored registered voice data. The electronic apparatus 100 may acquire a similarity between the received audio data and the registered voice data. When the similarity is greater than or equal to a threshold value, the electronic apparatus 100 may identify that the user who input the audio data is the authenticated user. When the similarity is greater than or equal to the threshold value, the electronic apparatus 100 may identify that the first user authentication is successful.

When the target user is identified as the authenticated user based on the audio data (S620-Y), the electronic apparatus 100 may display the first screen including the body part UI corresponding to the authenticated user (S630). The body part UI may be a pre-stored body part UI corresponding to the authenticated user. The body part UI may be a UI representing the body part of the user. The body part UI may be a UI generated based on the shape and size of the body part previously registered by the user.

The electronic apparatus 100 may acquire the touch data through the first screen (S635). While the body part UI is displayed through the first screen, the electronic apparatus 100 may receive the user touch. The user may input the user touch to an area corresponding to the location of the body part UI displayed on the display 140. The electronic apparatus 100 may acquire the touch data corresponding to the user touch through the touch sensor 185.

The electronic apparatus 100 may perform the second user authentication based on the touch data. The second user authentication may be touch authentication.

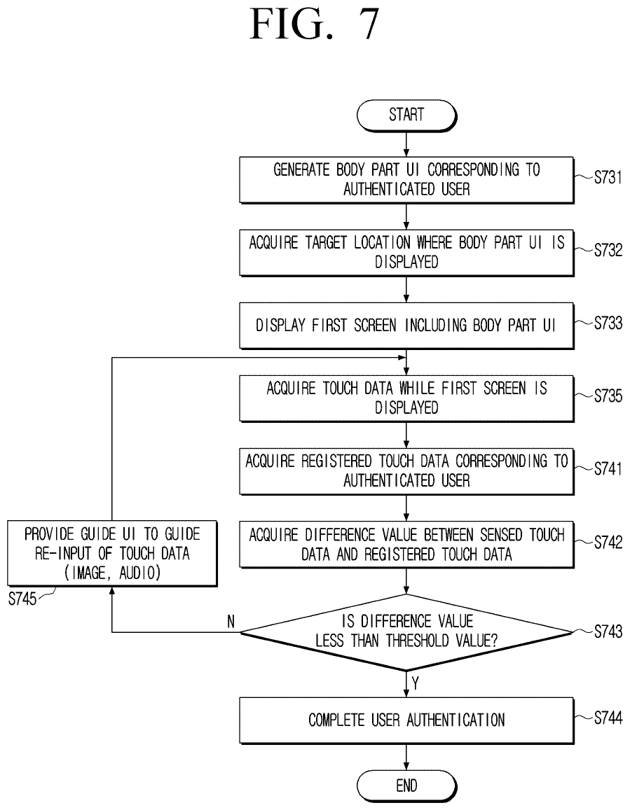

The electronic apparatus 100 may identify whether the user is the authenticated user based on the touch data (S640). The electronic apparatus 100 may perform the second user authentication by comparing the received touch data with the pre-stored registered touch data. The second user authentication is described in FIG. 7.

When the target user is identified as an authenticated user based on the touch data (S640-Y), the electronic apparatus 100 may acquire the user information corresponding to the authenticated user (S650). For example, the user information may include at least one of the body part information, the body analysis information, schedule information, preferred exercise information, or the history information.

The electronic apparatus 100 may identify the target exercise among multiple exercises based on the user information (S660). The target exercise may be a recommended exercise provided to the user.

The electronic apparatus 100 may display the second screen for guiding the target exercise (S670). The second screen may include the guide information for guiding the target exercise. The electronic apparatus 100 may generate the second screen for the target exercise determined based on the user information. The user may easily follow the target exercise through the second screen.

FIG. 7 is a diagram of a flowchart for describing the second user authentication operation according to an embodiment.

Referring to FIG. 7, the electronic apparatus 100 may generate the body part UI corresponding to an authenticated user (S731). The body part UI may be generated based on the body part information. The electronic apparatus 100 may generate the body part UI for authentication based on the pre-stored body part information.

For example, the electronic apparatus 100 may determine the target part based on the body part information. The electronic apparatus 100 may determine the target part among a plurality of parts included in the body part information. The target part may be a part for authentication. The electronic apparatus 100 may generate the body part UI corresponding to the target part among the plurality of parts.

The electronic apparatus 100 may acquire a target location at which to display the body part UI (S741). The target location may be a preset location. The target location may be changed according to the user's settings. The target location may correspond to a location where a touch sensor is disposed.

The electronic apparatus 100 may generate the first screen including the body part UI displayed at the target location.

The electronic apparatus 100 may display the first screen including the body part UI (S733). The body part UI may be displayed at the target location on the first screen.

The electronic apparatus 100 may acquire the touch data while the first screen is displayed (S735). The electronic apparatus 100 may acquire the registered touch data corresponding to the authenticated user (S741). The registered touch data may be data generated when the user has registered his or her touch data in advance.

The electronic apparatus 100 may acquire a difference value between the touch data (sensed touch data) obtained in operation S735 and the registered touch data (S742). The difference value may be described as a similarity. The electronic apparatus 100 may acquire the difference value by comparing the sensed touch data and the registered touch data.

For example, when the touch data is sensed in a capacitive manner, the difference value may represent an average value of the difference value of the capacitance at each of the plurality of locations.

When the difference value is less than the threshold value (S743-Y), the electronic apparatus 100 may identify that the user authentication is completed (S744).

When the difference value is greater than or equal to the threshold value (S743-N), the electronic apparatus 100 may provide the guide UI to guide the re-input of the touch data (S745). The guide UI may include at least one of the image data or the audio data. As an example, the guide UI may include “Not registered. Please re-touch.” FIG. 8 is a diagram of a flowchart for describing a location of a cable according to an embodiment.

Referring to FIG. 8, the electronic apparatus 100 may identify whether a grip is required in the target exercise (S871). The grip may indicate a hand grip connected to the electronic apparatus 100. When the grip is not required in the target exercise (S871-N), the electronic apparatus 100 may not provide a UI related to the grip.

When the grip is required in the target exercise (S871-Y), the electronic apparatus 100 may identify whether multiple grips are required in the target exercise (S872).

When multiple grips are not required in the target exercise (S872-N), the electronic apparatus 100 may identify the cable location corresponding to the target exercise based on the grip table for each exercise (S873).

The cable may connect the electronic apparatus 100 and the grip. The cable location table may include the location of the cable required for the exercise. The electronic apparatus 100 may identify the cable location corresponding to the target exercise among multiple positions based on the cable location table.

The electronic apparatus 100 may display the UI indicating the cable location (S874). The UI indicating the cable location may be generated based on a preset color or preset shape.

For example, assume that the grip is required for the first exercise. There may be a cable connecting the electronic apparatus 100 and the grip. When performing the first exercise, the cable may need to be disposed at the first location. The cable location table may include information mapping the first exercise and the first location. The electronic apparatus 100 may display a UI indicating the first location so that the cable may be placed at the first location when performing the first exercise.

When multiple grips are required for the target exercise (S872-Y), the electronic apparatus 100 may identify the first location of the first cable and the second location of the second cable corresponding to the target exercise based on the cable location table (S875).

The electronic apparatus 100 may display a UI indicating the first location and the second location (S876). The colors of the UI indicating the first location and the UI indicating the second location may be different.

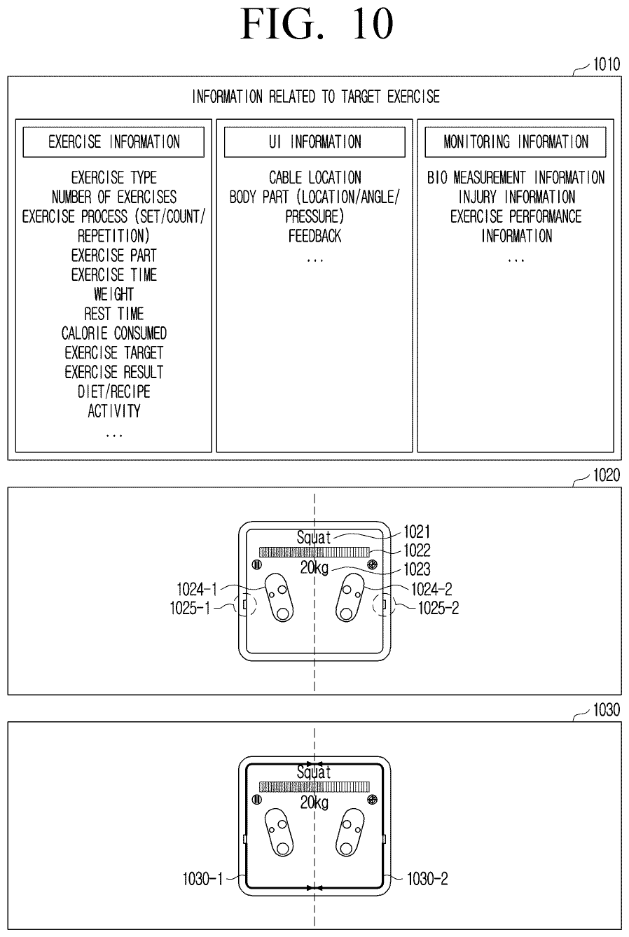

The description indicating the cable location is described in FIGS. 10 to 12.

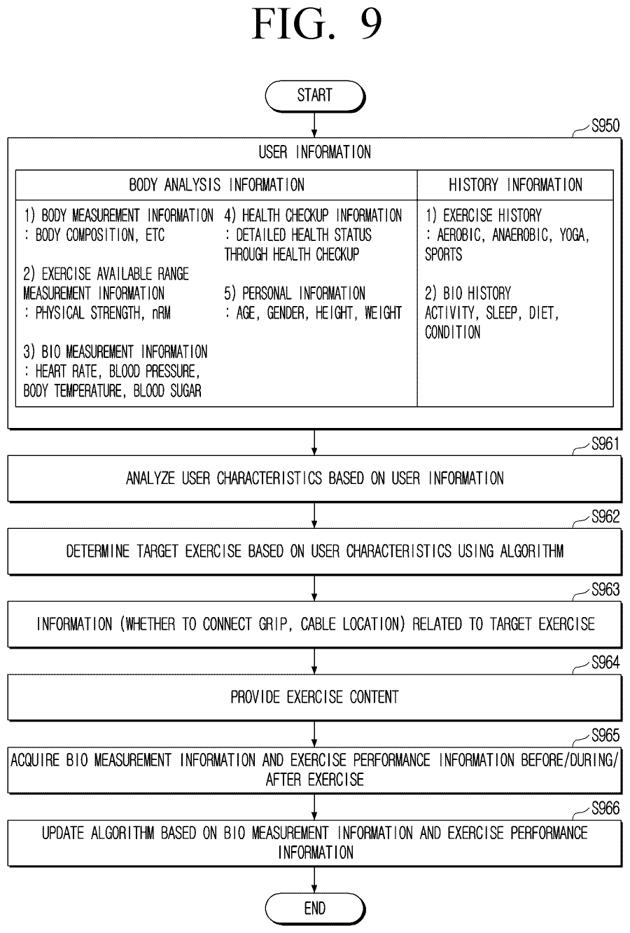

FIG. 9 is a diagram of a flowchart for describing an operation of acquiring the information related to the target exercise using the user information according to an embodiment.

Referring to FIG. 9, the electronic apparatus 100 may acquire the user information (S950). The electronic apparatus 100 may acquire the user information including the body analysis information and/or the history information. The operation of acquiring the user information may correspond to operation S650 of FIG. 6.

The electronic apparatus 100 may acquire the body analysis information among the user information. The body analysis information may include at least one of body measurement information, exercise available range measurement information, bio measurement information, health checkup information, or personal information.

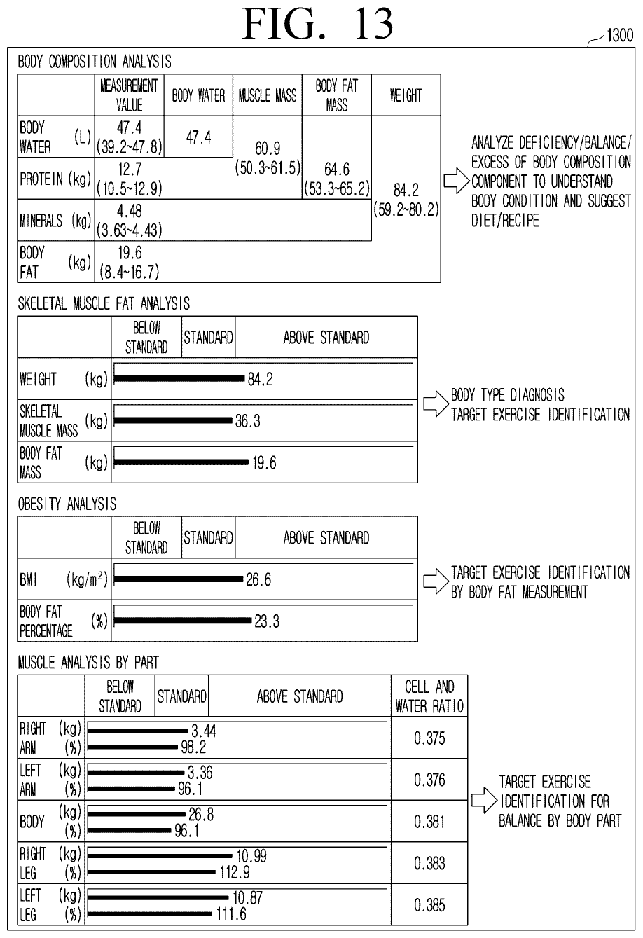

The body measurement information may include at least one of height, weight, body composition, skeletal muscle, fat, obesity, or muscle by area. A specific description related to the body measurement information is described in FIG. 13.

The exercise available range measurement information may include at least one of physical strength or n-repetition maximum (nRM).

The bio measurement information may include at least one of heart rate, blood pressure, body temperature, or blood sugar. For example, biometric information may be acquired through the terminal apparatus 300. The electronic apparatus 100 may acquire the biometric information from the terminal apparatus 300.

The health checkup information may include detailed health status through health checkup. The health checkup information may include information on underlying diseases.

The personal information may include at least one of age, gender, height, or weight.

The electronic apparatus 100 may acquire the history information among the user information. The history information may include at least one of exercise history or bio-history.

The exercise history may include at least one of the type of target exercise used by the electronic apparatus 100 or the exercise performance information.

The type of target exercise may indicate a specific exercise type such as aerobic, anaerobic, yoga, or sports.

The exercise performance information may indicate the degree of progress of the exercise. The exercise performance information may indicate whether the user has completed the exercise or whether the user has succeeded in all given exercises.

The bio-history may include a history related to a biorhythm of the user. For example, the bio-history may include at least one of activity, sleep, diet, or condition.

The electronic apparatus 100 may analyze user characteristics based on the user information including at least one of the body analysis information or the history information (S961).

The electronic apparatus 100 may determine the target exercise based on the analyzed user characteristics using an algorithm (S962). The electronic apparatus 100 may store an algorithm for determining the target exercise. The algorithm is a preset algorithm and may be changed according to the user's settings. The algorithm may be included in an artificial intelligence model. The electronic apparatus 100 may store the artificial intelligence model including an algorithm for determining the target exercise. The artificial intelligence model may be a model trained to determine the target exercise suitable for the user. When the user information is updated, the artificial intelligence model may also be updated. The update operation may include a learning operation.

When the target exercise is determined, the electronic apparatus 100 may acquire the information related to the target exercise (S963). The information related to the target exercise may include at least one of whether the grip should be connected to the target exercise or the cable location.

The electronic apparatus 100 may provide content for the target exercise. The electronic apparatus 100 may provide the exercise content (S964).

While providing the exercise content, the electronic apparatus 100 may acquire the biometric information and the exercise performance information (S965). The electronic apparatus 100 may acquire the biometric information and the exercise performance information at the time points before, during, and after the exercise. For example, the biometric information may be described as the condition information of the user. The exercise performance information may indicate whether the user performed the provided exercise content.

The electronic apparatus 100 may update an algorithm based on the acquired biometric information and the exercise performance information (S966). The updated algorithm may be used in an operation of determining the next target exercise.

For example, the user may not be able to complete the exercise content because the weight presented in the exercise content is too heavy. The electronic apparatus 100 may update the algorithm based on the exercise performance information indicating that the exercise content has not been completed. The algorithm may be trained to suggest a lower weight to the user.

FIG. 10 is a diagram for describing the information related to the target exercise according to an embodiment.

Referring to the embodiment 1010 of FIG. 10, the information related to the target exercise may be indicated. The information related to the target exercise may be information acquired in operation S963 of FIG. 9.

The information related to the target exercise may include at least one of exercise information, UI information, or monitoring information.

The exercise information may indicate various types of information related to the target exercise. For example, the exercise information may include at least one of exercise type, exercise number, set, number of times, repetition, exercise part, exercise time, weight, rest time, calories consumed, exercise goal, exercise result, diet, recipe, or activity amount.

The UI information may indicate information necessary for generating a UI displayed on a screen. The UI information may include at least one of cable location, body part location, body part angle, body part pressure, or feedback.

The monitoring information may indicate information that is subject to real-time measurement. The monitoring information may include at least one of bio-measurement information, injury information, or exercise performance information.

Referring to the embodiment 1020 of FIG. 10, the electronic apparatus 100 may provide the exercise content. The electronic apparatus 100 may display the second screen indicating the exercise content. The second screen may include at least one of an exercise type 1021, an exercise process 1022, a weight 1023, body parts 1024-1 and 1024-2, or cable locations 1025-1 and 1025-2. In one or more examples, the exercise content may be a sequence of still images showing motion corresponding to a desired exercise. In one or more examples, the exercise content may be a video showing the exercise, where the video may be repeated on a loop.

Referring to the embodiment 1030 of FIG. 10, the first cable connected to the electronic apparatus 100 may move along a first path 1030-1. The first path 1030-1 may indicate a movement path of the cable corresponding to a left area based on a central virtual line of the electronic apparatus 100. The second cable connected to the electronic apparatus 100 may move to a second path 1030-2. The second path 1030-2 may represent a movement path of the cable corresponding to the right area based on the central virtual line of the electronic apparatus 100.

For example, the first path 1030-1 and the second path 1030-2 may be described as a first rail and a second rail.

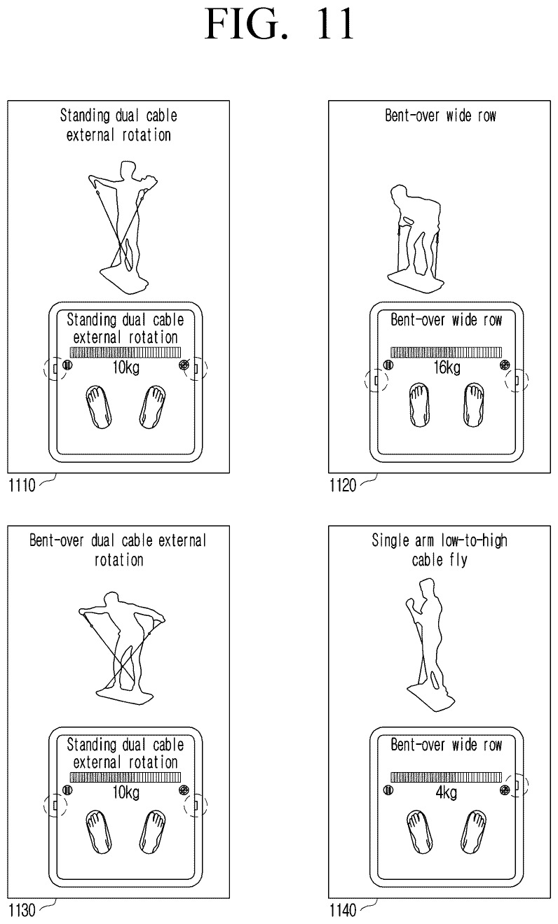

FIG. 11 is a diagram for describing a UI indicating a cable location according to an embodiment.

The cable location may be different depending on the type of target exercise. The display position of the UI indicating the cable location may also be different.

Referring to an embodiment 1110 of FIG. 11, the electronic apparatus 100 may provide the second screen corresponding to the target exercise (standing dual cable external rotation).

Referring to an embodiment 1120 of FIG. 11, the electronic apparatus 100 may provide the second screen corresponding to the target exercise (bent-over wide row).

Referring to an embodiment 1130 of FIG. 11, the electronic apparatus 100 may provide the second screen corresponding to the target exercise (bent-over dual cable external rotation).

Referring to an embodiment 1140 of FIG. 11, the electronic apparatus 100 may provide the second screen corresponding to the target exercise (single arm low-to-high cable fly).

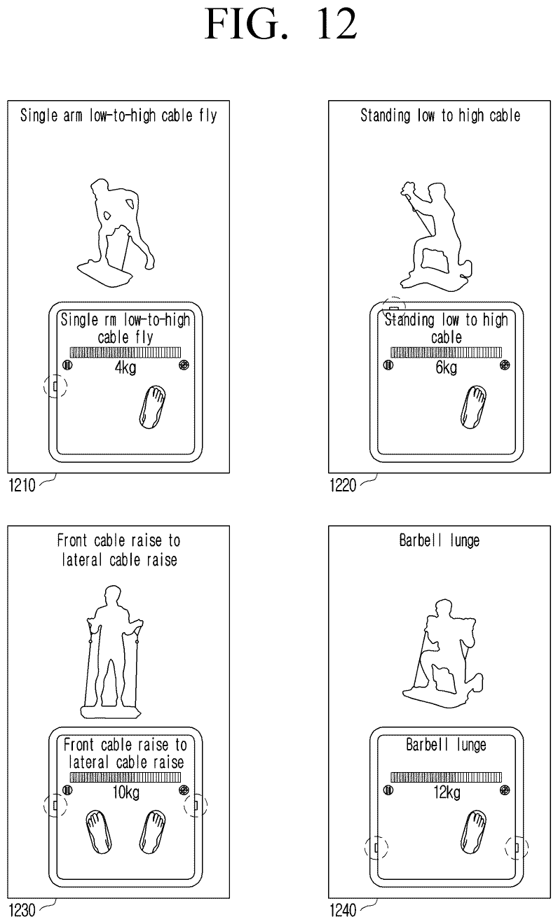

FIG. 12 is a diagram for describing a UI indicating a cable location according to an embodiment.

The cable location may be different depending on the type of target exercise. The display location of the UI indicating the cable location may also be different.

Referring to an embodiment 1210 of FIG. 12, the electronic apparatus 100 may provide the second screen corresponding to the target exercise (single arm low-to-high cable fly).

Referring to the embodiment 1220 of FIG. 12, the electronic apparatus 100 may provide the second screen corresponding to the target exercise (standing low to high cable).

Referring to the embodiment 1230 of FIG. 12, the electronic apparatus 100 may provide the second screen corresponding to the target exercise (front cable raise to lateral cable raise).

Referring to the embodiment 1240 of FIG. 12, the electronic apparatus 100 may provide the second screen corresponding to the target exercise (barbell lunge).

FIG. 13 is a diagram for describing an operation of analyzing user's body measurement information 1300 according to an embodiment.

Referring to FIG. 13, the electronic apparatus 100 may include body measurement information 1300. The body measurement information 1300 may include at least one of body composition analysis, skeletal muscle analysis, fat analysis, obesity analysis, or muscle analysis by area.

The electronic apparatus 100 may acquire body composition components based on body composition analysis. The electronic apparatus 100 may identify a user's body condition based on the body composition components. The electronic apparatus 100 may identify a diet or recipe according to the body condition.

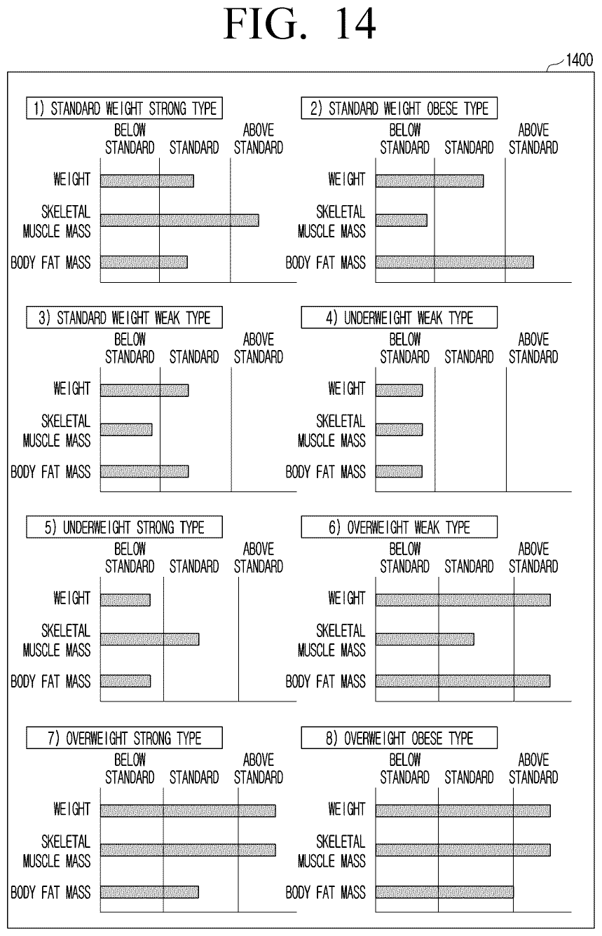

The electronic apparatus 100 may determine a user's body type based on skeletal muscle analysis and fat analysis. The electronic apparatus 100 may identify the target exercise based on the user's body type. The information related to the body type is described in FIG. 14.

The electronic apparatus 100 may acquire a body fat percentage based on obesity analysis. The electronic apparatus 100 may determine the target exercise based on the body fat percentage.

The electronic apparatus 100 may identify target exercises for balance of each body part based on muscle analysis of each body part.

FIG. 14 is a diagram for describing the distinguished body types according to an embodiment.

Referring to an embodiment 1400 of FIG. 14, the electronic apparatus 100 may identify the body type of the user based on skeletal muscle analysis and fat analysis. The body type may be a preset type.

For example, the preset types may include at least one of a standard weight strong type, a standard weight obese type, a standard weight weak type, a low weight weak type, a low weight strong type, a low weight weak type, an overweight strong type, or an overweight obese type. The body type analysis may be based on an image of the user.

FIG. 15 is a diagram for describing guide information according to body type according to an embodiment.

A table 1500 of FIG. 15 may include guide information according to body type. The table 1500 may include the target exercise corresponding to the body type or diets corresponding to the body type.

The table 1500 may include target exercises corresponding to each body type described in FIG. 14 and the diets corresponding to each body type.

For example, when the body type is determined, the electronic apparatus 100 may identify the target exercise corresponding to the body type based on the table 1500.

For example, when the body type is determined, the electronic apparatus 100 may identify the diets corresponding to the body type based on the table 1500.

For example, when the body type is determined, the electronic apparatus 100 may identify the target exercises and diets corresponding to the body type based on the table 1500.

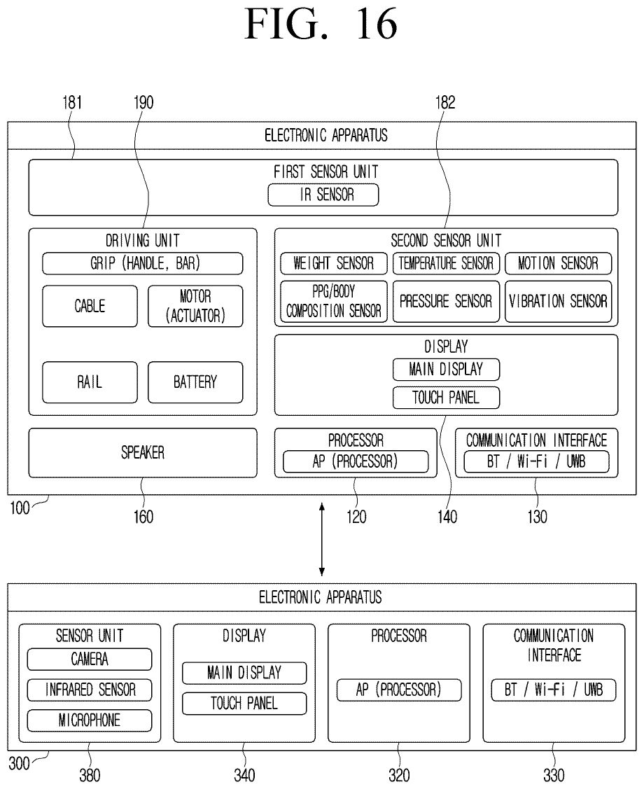

FIG. 16 is a diagram for describing the structure of the electronic apparatus and the terminal apparatus according to an embodiment.

The system 1600 of FIG. 16 may include at least one of an electronic apparatus 100 or the terminal apparatus 300.

The electronic apparatus 100 may include at least one processor 120, a communication interface 130, a display 140, a speaker 160, a first sensor unit 181, a second sensor unit 182, and a driving unit 190. In one or more examples, each unit may be implemented by individual circuitry, one or more processors executing one or more instructions, or a combination of circuitry and one or more processors executing one or more instructions.

At least one processor 120 may include an application processor (AP).

The communication interface 130 may include at least one of a Bluetooth module, a WiFi module, or an ultra-wideband (UWB) module.

The display 140 may include at least one of a main display or a touch panel.

The speaker 160 may output guide audio.

The first sensor unit 181 may include an infrared sensor.

The second sensor unit 182 may include at least one of a weight sensor, a temperature sensor, a motion sensor, a photoplethysmography (PPG) sensor, a body composition sensor, a pressure sensor, and a vibration sensor. The driving unit 190 may be connected to at least one of the grip, the cable, the motor, the rail, or the battery.

The driving unit 190 may provide physical force generated by the motor to at least one of the grip, the cable, or the rail. The electronic apparatus 100 may supply power stored in the battery to the motor. The electronic apparatus 100 may drive the motor and provide physical force to the cable.

The terminal apparatus 300 may include at least one of a processor 320, a communication interface 330, a display 340, or a sensor unit 380.

At least one of the processor 320 may include the application processor (AP).

The communication interface 330 may include at least one of the Bluetooth module, the WiFi module, of the ultra-wideband (UWB) module.

The display 340 may include at least one of the main display or the touch panel.

The sensor unit 380 may include at least one of the camera, the infrared sensor, or the microphone.

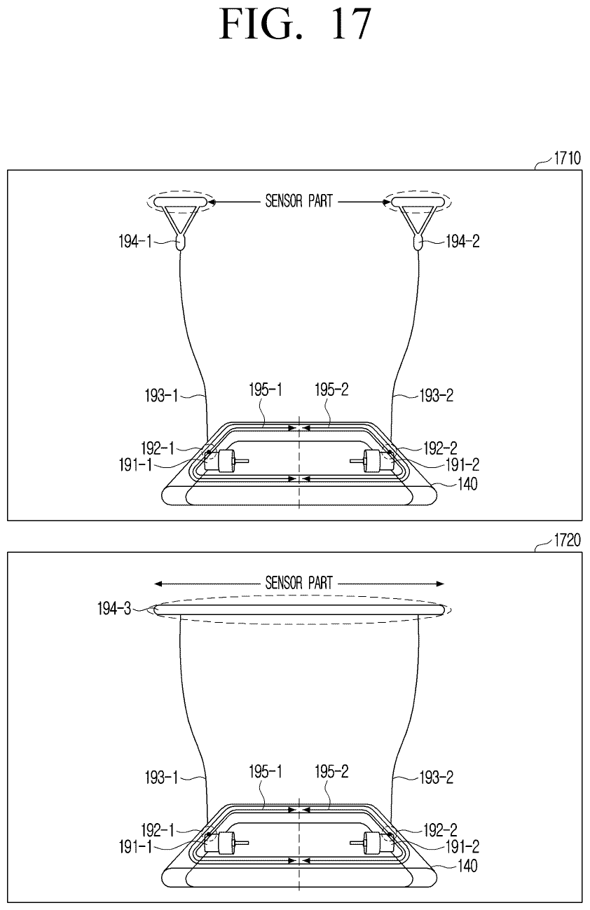

FIG. 17 is a diagram for describing a grip connected to an electronic apparatus according to an embodiment.

Referring to an embodiment 1710 of FIG. 17, the electronic apparatus 100 may be connected to grips 194-1 and 194-2 in the form of a handle.

The electronic apparatus 100 may include a first motor 191-1, a first cable 193-1, and a first grip 194-1. The first motor 191-1 may be connected to the first cable 193-1. The first motor 191-1 may be arranged at a lower side of the display 140. The first cable 193-1 may move on a first path 195-1. The first cable 193-1 may be connected to the first grip 194-1. A sensor may be arranged on a handle portion of the first grip 194-1. The force for pulling the first grip 194-1 may be determined by the physical force provided by the first motor 191-1. The electronic apparatus 100 may identify the first position 192-1 of the first cable 193-1. The electronic apparatus 100 may provide a UI indicating the first position 192-1.

The electronic apparatus 100 may include a second motor 191-2, a second cable 193-2, and a second grip 194-2. The second motor 191-2 may be connected to the second cable 193-2. The second motor 191-2 may be arranged at the lower side of the display 140. The second cable 193-2 may move on a second path 195-2. The second cable 193-2 may be connected to the second grip 194-2. A sensor may be placed on the handle portion of the second grip 194-2. The force for pulling the second grip 194-2 may be determined by the physical force provided by the second motor 191-2. The electronic apparatus 100 may identify the second position 192-2 of the second cable 193-2. The electronic apparatus 100 may provide a UI indicating the second position 192-2.

An embodiment 1720 of FIG. 17 may correspond to the embodiment 1710. Duplicate descriptions will be omitted. The electronic apparatus 100 may be connected to a third grip 194-3 in the form of a bar instead of the first and second grips 194-1 and 194-2 in the form of a handle. The touch sensors may be arranged throughout the bar of the third grip 194-3.

The grips 194-1, 194-2, and 194-3 connected to the electronic apparatus 100 may include sensors.

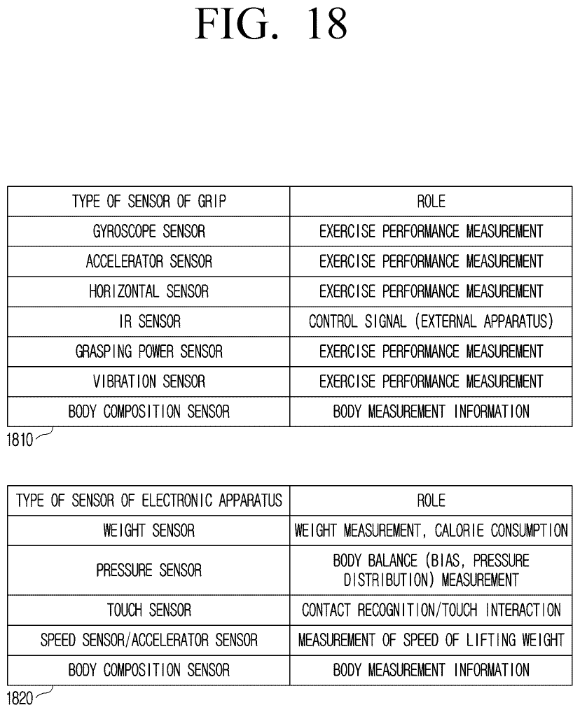

FIG. 18 is a diagram for describing a plurality of sensors included in the grip and the electronic apparatus according to an embodiment.

A table 1810 of FIG. 18 may represent sensors arranged in the grips 194-1, 194-2, and 194-3. The grips 194-1, 194-2, and 194-3 may include at least one of a gyroscope sensor, an acceleration sensor, a horizontal sensor, an infrared sensor, a grip force sensor, a vibration sensor, or a body composition sensor.

The electronic apparatus 100 may acquire the sensing data obtained from the sensors included in the grips 194-1, 194-2, and 194-3.

The electronic apparatus 100 may measure the user's exercise performance based on the sensing data acquired from at least one sensor among the gyroscope sensor, the acceleration sensor, the horizontal sensor, the grip force sensor, and the vibration sensor. The exercise performance may include information indicating whether the user is performing the exercise correctly.

The electronic apparatus 100 may receive a control signal from an external apparatus using the infrared sensor. The external device may include the terminal apparatus 300 or the remote control apparatus.

The electronic apparatus 100 may acquire the body measurement information based on the sensing data acquired through the body composition sensor.

A table 1820 of FIG. 18 may represent sensors arranged in the electronic apparatus 100. The electronic apparatus 100 may include at least one of a weight sensor, a pressure sensor, a touch sensor, a speed sensor, an acceleration sensor, or a body composition sensor.

The electronic apparatus 100 may identify at least one of the user's body weight or calorie consumption based on the sensing data acquired through the weight sensor.

The electronic apparatus 100 may measure body balance based on the sensing data acquired through a pressure sensor. The body balance may include at least one of bias or pressure distribution.

The electronic apparatus 100 may recognize a user's contact or identify a touch interaction based on the sensing data acquired through a touch sensor.

The electronic apparatus 100 may measure a speed of lifting a weight based on the sensing data acquired through the speed sensor or the acceleration sensor.

The electronic apparatus 100 may acquire the body measurement information based on the sensing data acquired through the body composition sensor.

For example, both the grips 194-1, 194-2, and 194-3 and the electronic apparatus 100 may include the body composition sensor. The electronic apparatus 100 may acquire the body measurement information based on the sensing data acquired from the grips 194-1, 194-2, and 194-3 and the sensing data acquired from the sensor included in the table 1820.

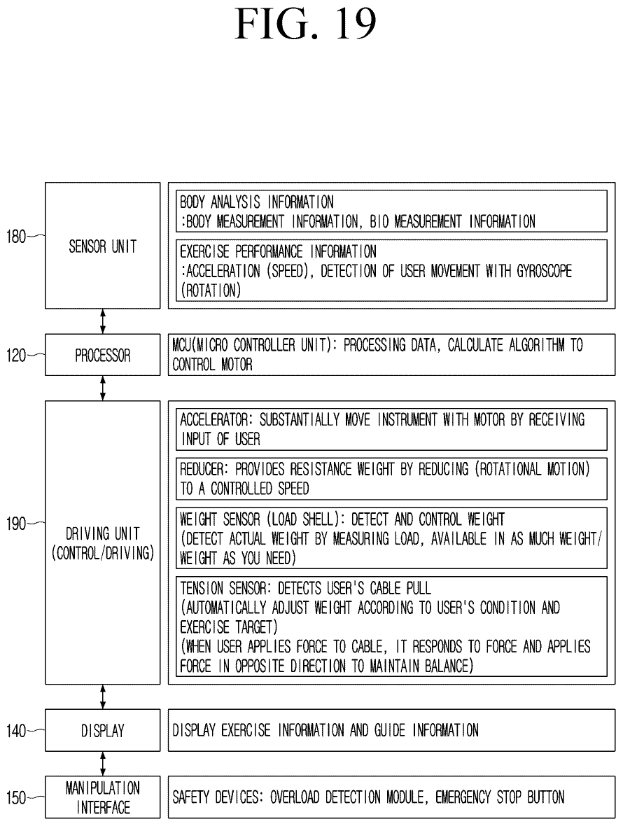

FIG. 19 is a diagram for describing the operation performed by each component in the electronic apparatus according to an embodiment.

Referring to FIG. 19, the electronic apparatus 100 may include at least one of the sensor unit 180, at least one processor 120, the driving unit 190, the display 140, or the manipulation interface 150.

The electronic apparatus 100 may acquire the sensing data through the sensor unit 180. The electronic apparatus 100 may acquire the body analysis information based on the sensing data. The body analysis information may include at least one of body measurement information or the biometric information.

The electronic apparatus 100 may acquire the exercise performance information based on the sensing data. The electronic apparatus 100 may acquire the exercise performance information including user movement based on the sensing data. The sensor unit 180 may include at least one of the acceleration sensor, the speed sensor, or the gyroscope sensor. The sensor unit 180 may detect user movement based on the sensing data acquired from at least one of the acceleration sensor, the speed sensor, or the gyroscope sensor.

At least one processor 120 may include a micro controller unit (MCU). The MCU may perform an operation of processing the sensing data acquired from the sensor unit 180. The MCU may perform an operation of controlling a motor (e.g., an actuator) that provides power to the cable. The MCU may use a preset control algorithm.

The driving unit 190 may include at least one of an accelerator, a reducer, a weight sensor, or a tension sensor.

The accelerator may receive a user's input and control the motor to move the cable.

The reducer may provide a resistive weight to reduce the speed according to a preset control signal.

The weight sensor (e.g., a load shell) may be a sensor that detects and adjusts weight. The weight sensor may measure a weight of a load. The electronic apparatus 100 may provide the required weight (or weight) based on the sensing data acquired by the weight sensor.

The tension sensor may detect the user's pulling of the cable. The electronic apparatus 100 may sense the force that the user pulls the cable based on the sensing data acquired through the tension sensor. The electronic apparatus 100 may set the user's status and exercise goal based on the sensing data. The electronic apparatus 100 may adjust a weight. The electronic apparatus 100 may maintain balance by providing force in the opposite direction of the force that the user applies to the cable based on the sensing data.

At least one processor 120 may process various data acquired from the driving unit 190.

The display 140 may display the exercise content including at least one of the exercise information or guide information.

The operating interface 150 may include a stabilizing device. The stabilizing device may include at least one of a device for preventing overload or an emergency stop button. When the force applied to the electronic apparatus 100 is greater than a threshold value, the device for preventing overload may stop the function of providing the exercise content. When a user input for pressing an emergency stop button is received, the electronic apparatus 100 may stop the function of providing the exercise content.

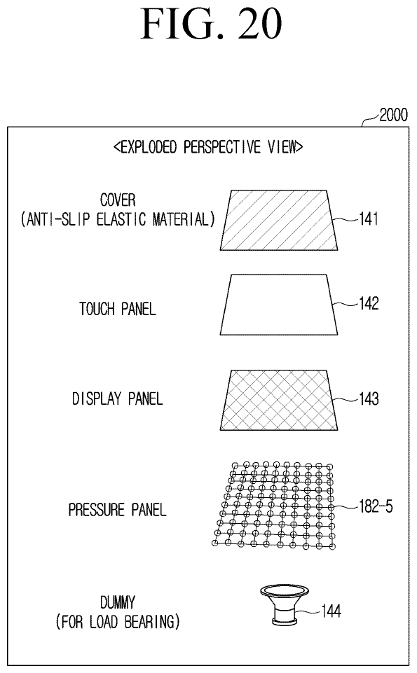

FIG. 20 is a diagram for describing the structure of the electronic apparatus according to an embodiment.

Referring to an embodiment 2000 of FIG. 20, the structure of the display 140 may be illustrated. The display 140 may be implemented as a display module. The display module may include at least one of a cover 141, a touch panel 142, a display panel 143, a pressure sensor 182-5, or a dummy 144.

The cover 141 may be implemented as an elastic material to prevent slipping.

The touch panel 142 may be a panel for detecting a user touch. The touch panel 142 may include a touch sensor. The electronic apparatus 100 may recognize a touch location based on the touch data acquired through the touch sensor.

The display panel 143 may include an optical element that outputs a pixel value.

The pressure sensor 182-5 may be a sensor that detects the user touch pressure. For example, the pressure sensor 182-5 may be a sensor included in the second sensor unit 182 of FIG. 16. The electronic apparatus 100 may acquire a pressure value for each touch location based on the sensing data (or pressure data) obtained through the pressure sensor 182-5.

The dummy 144 may be a member for supporting a load.

FIG. 21 is a diagram for describing an operation of recognizing a user touch according to an embodiment.

Referring to an embodiment 2110 of FIG. 21, the electronic apparatus 100 may acquire a pressure distribution graph based on the touch data and the pressure data.

The electronic apparatus 100 may receive the touch data and pressure data for the user touch. It is assumed that the user has placed both feet on the electronic apparatus 100. However, in one or more examples, the touch data and the pressure data may correspond to a user pressing a finger or hand on a screen of the electronic apparatus 100. The electronic apparatus 100 may acquire the touch data through the touch sensor included in the sensor unit 180. The electronic apparatus 100 may acquire the pressure data through the pressure sensor included in the sensor unit 180. The touch data may indicate a location where the user touches the display 140 of the electronic apparatus 100. The pressure data may indicate a distribution of pressure that the user applies to the electronic apparatus 100. A pressure sensing value may be higher in an area where a load is applied. The electronic apparatus 100 may acquire a pressure distribution graph based on the touch data and the pressure data.

For example, the electronic apparatus 100 may acquire a pressure distribution graph using only the pressure data.

Referring to an embodiment 2120 of FIG. 21, the electronic apparatus 100 may provide a guide UI for adjusting the user's foot position. For example, the electronic apparatus 100 may provide a guide UI for guiding the user to adjust the user's foot position. For example, the electronic apparatus 100 may provide a guide UI requesting to release the force from the heel. The electronic apparatus 100 may acquire the touch data indicating the pressure distribution for the user's load using a pressure sensor.

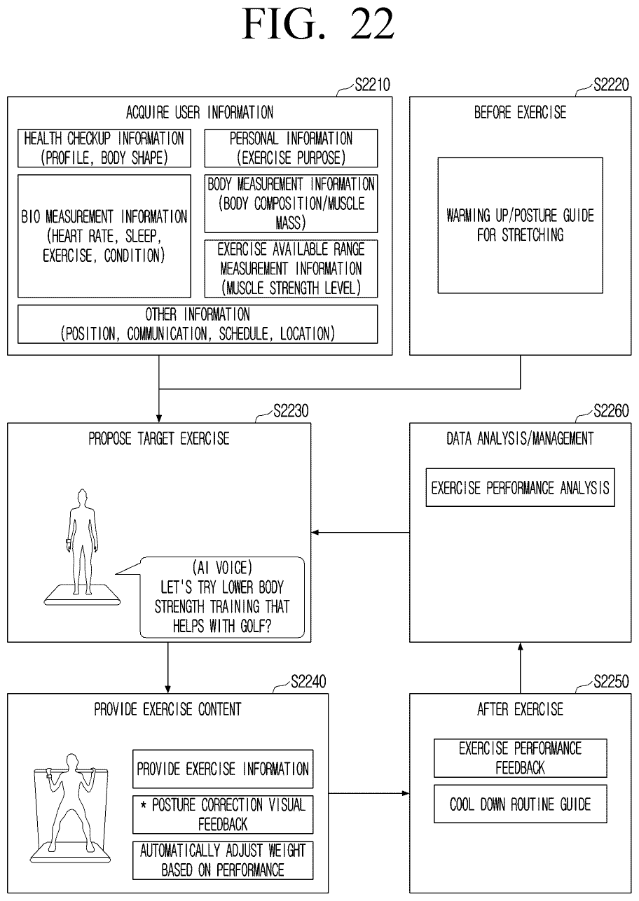

FIG. 22 is a diagram for describing an operation of guiding a user to the target exercise according to an embodiment.

Referring to FIG. 22, the electronic apparatus 100 may acquire the user information (S2210). The user information may include at least one of health checkup information (profile, body type, etc.), personal information (exercise information, etc.), the biometric information (heart rate, sleep, exercise, condition, etc.), body measurement information (body composition, muscle mass, etc.), exercise available range measurement information (muscle strength level, etc.), or other information (location, communication, schedule, place, etc.).

The electronic apparatus 100 may provide first content for warming up or stretching before providing the exercise content (S2220). The first content may be a posture guide content for warming up or stretching. The electronic apparatus 100 may collect (or obtain) the user information while providing the first content.

The electronic apparatus 100 may suggest the target exercise (S2230). The electronic apparatus 100 may determine the target exercise based on the user information. When the first content ends, the electronic apparatus 100 may determine the target exercise based on the user information. The electronic apparatus 100 may provide a UI (Shall we try lower body strength training that helps with golf?) to notify that the target exercise will be provided. The UI may be provided as at least one of image data or the audio data.

The electronic apparatus 100 may provide the exercise content. The exercise content may be described as second content. The electronic apparatus 100 may provide second content related to the target exercise. The electronic apparatus 100 may provide the exercise information. The exercise information may include an exercise name and guide information indicating an exercise posture. The electronic apparatus 100 may provide visual feedback for posture correction. The electronic apparatus 100 may acquire the exercise performance information. The electronic apparatus 100 may automatically adjust the weight based on the exercise performance information.

When the exercise content (second content) ends, the electronic apparatus 100 may acquire the exercise performance information. The exercise performance information obtained in operation S2240 is information collected in real time, and the exercise performance information obtained in operation S2250 may be result data that synthesizes the information collected in real time. The electronic apparatus 100 may provide feedback based on the exercise performance information.

When the exercise content (second content) ends, the electronic apparatus 100 may provide third content to guide a cool-down routine. The cool-down routine may represent a cool-down exercise that relaxes muscles. For example, the first content and the third content may be different.

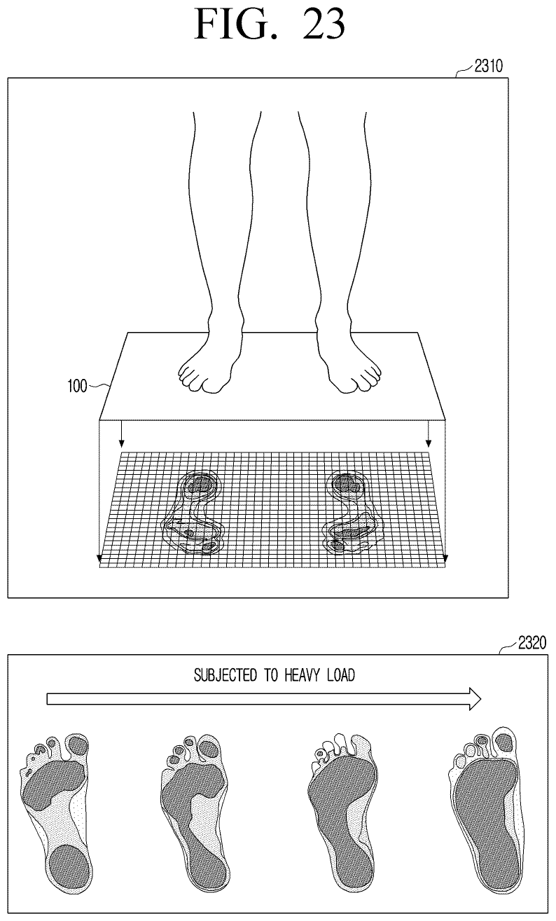

FIG. 23 is a diagram for describing an operation of recognizing the user touch and performing the second user authentication according to an embodiment.

Referring to the embodiment 2310 of FIG. 23, the electronic apparatus 100 may acquire the user's touch data and pressure data. The electronic apparatus 100 may acquire a pressure distribution graph based on the touch data and the pressure data. The pressure distribution graph may represent the pressure distribution for the entire touch area. The pressure distribution graph may represent which area is subjected to a large load due to the user touch.

Referring to the embodiment 2320 of FIG. 23, the electronic apparatus 100 may represent a situation in which a load is applied differently even when the same user touches. The pressure value may be higher as the load is applied more. The higher the pressure value, the darker the pixel may be represented. The load may be represented as the load is applied more as the graph goes to the right. For example, the user may increase the load in the direction of gravity (downward direction) by pulling the grab. As the load increases, the pressure value for more areas may increase.

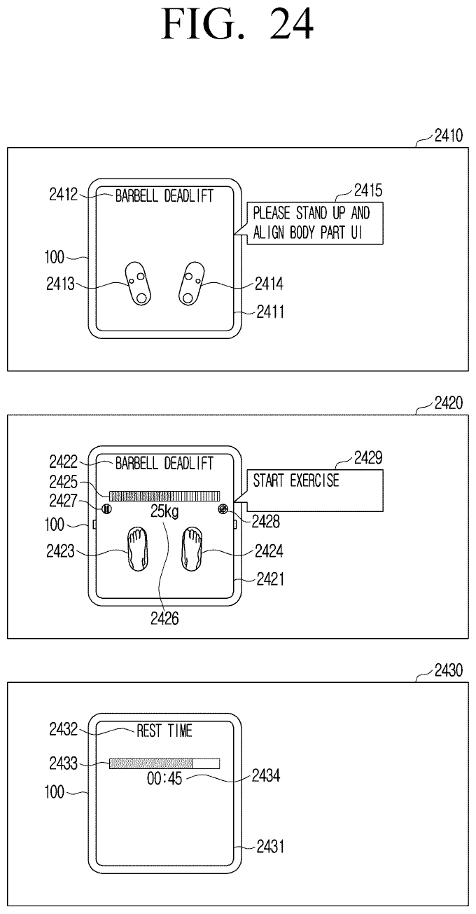

FIG. 24 is a diagram for describing an operation of providing a guide UI related to exercise according to an embodiment.

Referring to the embodiment 2410 of FIG. 24, the electronic apparatus 100 may display the first screen 2411. For example, when a user is identified as an authenticated user based on the audio data according to operation S620 of FIG. 6, the electronic apparatus 100 may display the first screen 2411.

The first screen 2411 may include at least one of a UI 2412 indicating the type (or name) of the target exercise and the first body part UI 2413 and 2414. The electronic apparatus 100 may provide a UI 2415 for requesting the user to move in accordance with the first body part UI 2413 and 2414 while displaying the first screen 2411. For example, the UI 2415 may be provided in the form of the audio data.

Referring to the embodiment 2420 of FIG. 24, the electronic apparatus 100 may display the second screen 2421 for providing the exercise content. For example, when the user is identified as an authenticated user based on the touch data according to operation S640 of FIG. 6, the electronic apparatus 100 may display the second screen 2421.

The second screen 2421 may include at least one of a UI 2412 indicating the type (or name) of the target exercise, the second body part UI 2423 and 2424, a UI 2425 indicating the progress of the target exercise, a UI 2426 indicating the weight of the target exercise, a UI 2427 for controlling playback and pause of content, or a UI 2428 for controlling display and hiding of guide information.

The electronic apparatus 100 may generate the second screen 2421 including UIs 2422, 2423, 2424, 2425, 2426, 2427, and 2428. The electronic apparatus 100 may display the generated second screen 2421.

While the second screen 2421 is displayed, the electronic apparatus 100 may provide a UI 2429 to notify that the exercise content is provided. For example, the UI 2429 may be provided in the form of the audio data.

Referring to the embodiment 2430 of FIG. 24, the electronic apparatus 100 may display a third screen 2431 related to a break time. While providing the exercise content, the electronic apparatus 100 may provide information to notify the user of the break time. When an event for providing a break time is identified while providing the exercise content, the electronic apparatus 100 may display a third screen 2431.

The third screen 2431 may include at least one of a UI 2432 indicating a break time, a UI 2433 indicating the progress of the remaining break time, of a UI 2434 indicating the remaining break time.

FIG. 25 is a diagram for describing an embodiment of providing a guide screen to a user according to an embodiment.

Referring to an embodiment 2510 of FIG. 25, the electronic apparatus 100 may provide a UI 2511 that inquires whether to provide the exercise content. For example, the electronic apparatus 100 may provide a UI 2511 that includes “Today, a golf exercise is scheduled at 3 PM at home. Would you like to exercise?” The UI 2511 may be provided as at least one of image data or the audio data. In response to the provided UI 2511, the user may input a control command requesting the exercise content. The electronic apparatus 100 may receive a control command (or user input) requesting the exercise content from the user.

Referring to an embodiment 2520 of FIG. 25, after a user input requesting the exercise content is received, the electronic apparatus 100 may provide a UI 2521 for suggesting the target exercise. For example, the electronic apparatus 100 may provide a UI 2521 including “A total of 18 minutes will be required for 4 exercises for golf swing. Are you ready? ” The UI 2521 may be provided as at least one of image data or the audio data. In response to the provided UI 2521, the user may input a control command requesting the start of the exercise content. The electronic apparatus 100 may receive a control command (or user input) requesting the start of the exercise content from the user.

Referring to the embodiment 2530 of FIG. 25, after the control command requesting the start of the exercise content is received, the electronic apparatus 100 may display the first screen 2531 for providing the exercise content. The first screen 2531 may include the first body part UIs 2532 and 2533. The user may touch a part of the user's body to the electronic apparatus 100 through the first body part UIs 2532 and 2533.

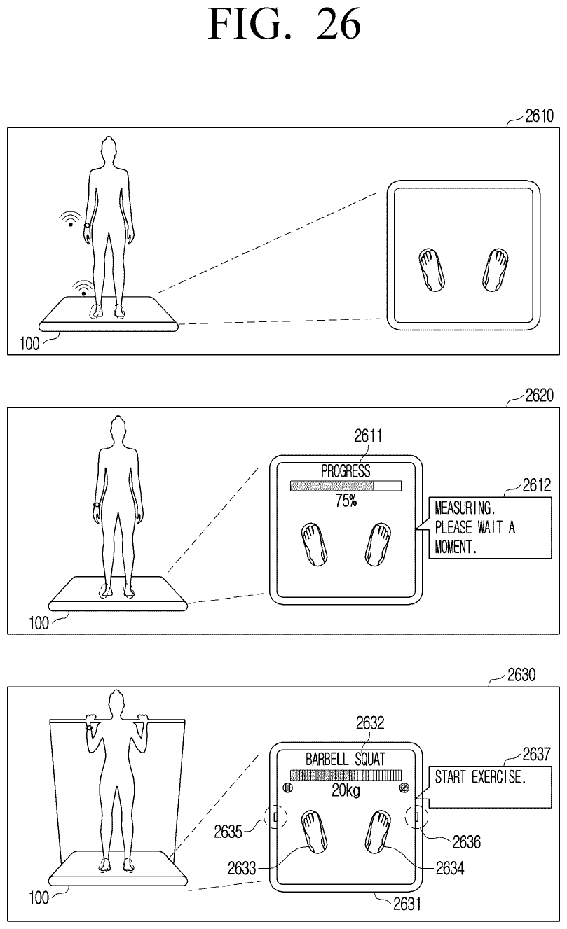

FIG. 26 is a diagram for describing an embodiment of providing a guide screen to the user according to an embodiment.

Referring to an embodiment 2610 of FIG. 26, it is assumed that the user is standing on the electronic apparatus 100 after the first screen 2531 of FIG. 25 is displayed.

Referring to an embodiment 2620 of FIG. 26, the electronic apparatus 100 may perform the second user authentication. According to the embodiment 2610, when the user is standing on the electronic apparatus 100, the electronic apparatus 100 may acquire the touch data. The electronic apparatus 100 may perform the second user authentication based on the touch data. While performing the second user authentication, the electronic apparatus 100 may provide UIs 2611 and 2612 related to the second user authentication. The UI 2611 may indicate the progress of the second user authentication. The UI 2612 may indicate that the second user authentication is in progress. The UI 2612 may include information guiding the user to maintain a posture.

Referring to an embodiment 2630 of FIG. 26, when the second user authentication is successful, the electronic apparatus 100 may display the second screen 2631 for the exercise content. The electronic apparatus 100 may provide the exercise content for providing the target exercise. The electronic apparatus 100 may display the second screen 2631 for the exercise content.

The second screen 2631 may include at least one of a UI 2632 related to target exercise, second body part UIs 2633 and 2364, or UIs 2635 and 2636 indicating a cable location.

The UI 2632 may include at least one of the name of the target exercise, the progress of the target exercise, or the weight of the target exercise.

The second body part UIs 2633 and 2634 may indicate the position of the body part to guide the posture of the target exercise.

The UIs 2635 and 2636 may indicate the cable location connected to the grip required for the target exercise.

The electronic apparatus 100 may provide a UI 2637 to notify the provision of the exercise content related to the target exercise.

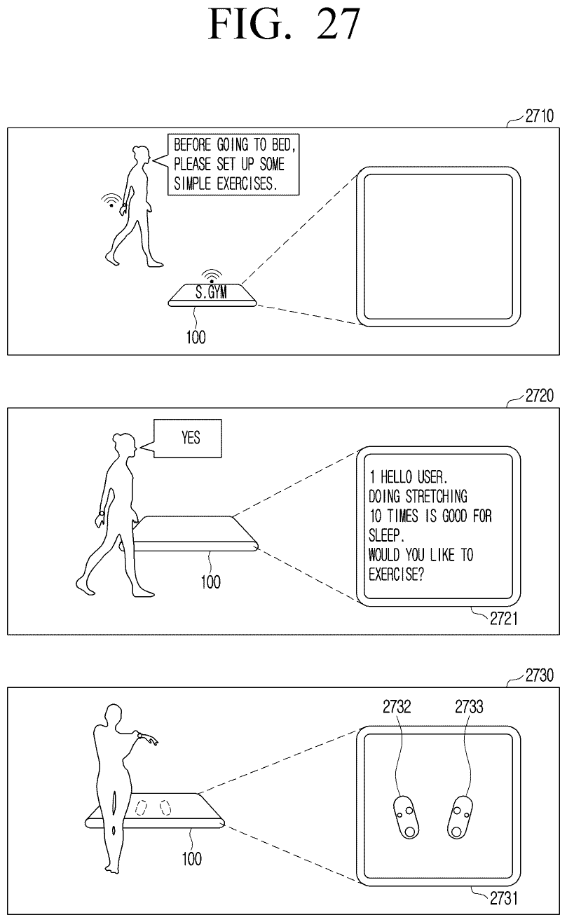

FIG. 27 is a diagram for describing an embodiment in which a user requests a guide screen according to an embodiment.

Referring to an embodiment 2710 of FIG. 27, a user may first request the electronic apparatus 100 to provide the exercise content. The electronic apparatus 100 may receive a control command (or user input) requesting the exercise content from the user. As an example, the electronic apparatus 100 may receive the control command through the user's voice (Set it as a simple exercise before going to sleep).

Referring to an embodiment 2720 of FIG. 27, when a control command requesting the exercise content is received, the electronic apparatus 100 may determine the target exercise. The electronic apparatus 100 may provide a UI 2721 to guide the target exercise. For example, the UI 2721 may include “When you do stretching 10 times, it is good for sleep. Would you like to exercise?” The UI 2721 may be provided as at least one of image data or the audio data. In response to the provided UI 2721, the user may input a control command requesting the start of the exercise content. The electronic apparatus 100 may receive a control command (or user input) requesting the start of the exercise content from the user.

Referring to an embodiment 2730 of FIG. 27, after the control command requesting the start of the exercise content is received, the electronic apparatus 100 may display the first screen 2731 for providing the exercise content. The first screen 2731 may include first body part UIs 2732 and 2733. The user may touch a part of the user's body to the electronic apparatus 100 through the first body parts UI 2732 and 2733.

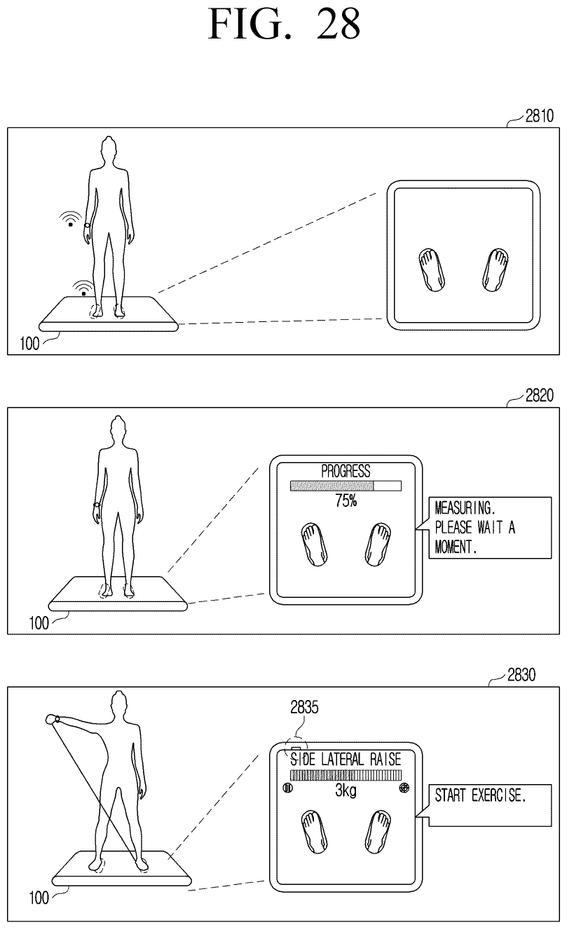

After the embodiment 2730 of FIG. 27, the embodiment 2810 of FIG. 28 may be performed.

FIG. 28 is a diagram for describing an embodiment in which a user requests a guide screen according to an embodiment.

An embodiment 2810 of FIG. 28 may correspond to the embodiment 2610 of FIG. 26. The embodiment 2820 of FIG. 28 may correspond to the embodiment 2620 of FIG. 26. The embodiment 2830 of FIG. 28 may correspond to the embodiment 2630 of FIG. 26. Duplicate descriptions are omitted.

An embodiment 2830 of FIG. 28 may have a different target exercise than the embodiment 2630 of FIG. 26. In the embodiment 2830, the target exercise may use only one grip. When the target exercise requires one grip, only one cable may be used. The electronic apparatus 100 may display a UI 2835 indicating the position of one grip.

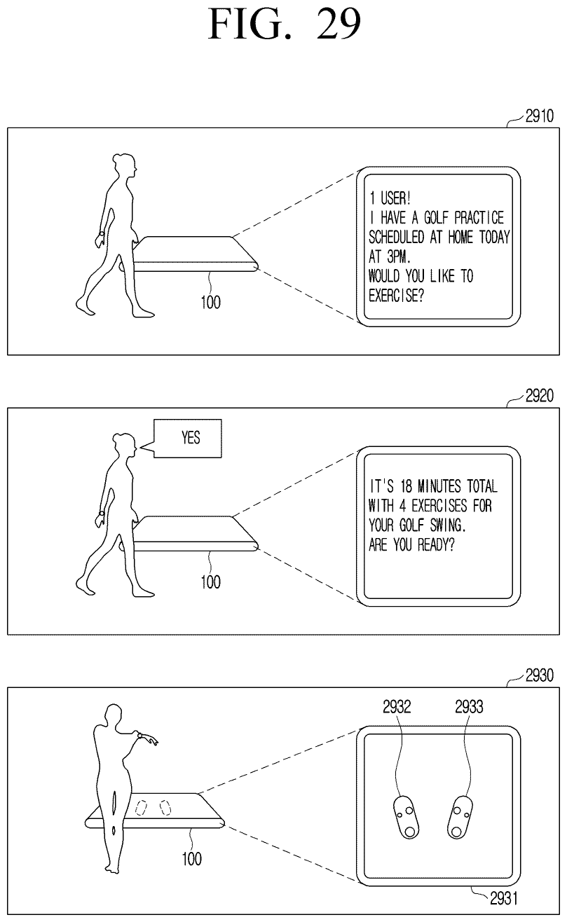

FIG. 29 is a diagram for describing an embodiment in which a user is replaced according to an embodiment.

An embodiment 2910 of FIG. 29 may correspond to the embodiment 2510 of FIG. 25. The embodiment 2920 of FIG. 29 may correspond to the embodiment 2520 of FIG. 25. The embodiment 2930 of FIG. 29 may correspond to the embodiment 2530 of FIG. 25. Duplicate descriptions are omitted.

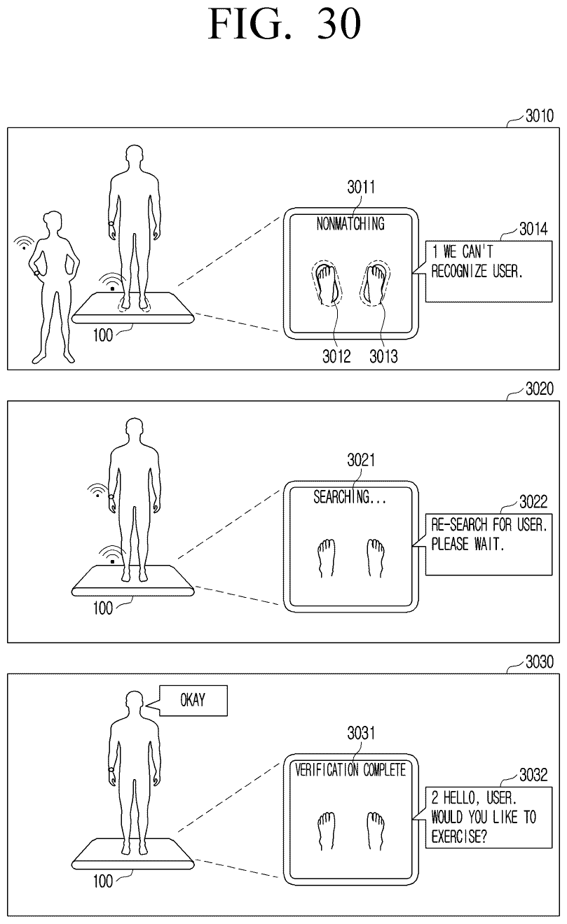

FIG. 30 is a diagram for describing an embodiment in which a user is replaced according to an embodiment.

Referring to an embodiment 3010 of FIG. 30, it is assumed that a new user, not a user for the first user authentication in the embodiment 2930 of FIG. 29, is standing on the electronic apparatus 100. In the embodiment 2930 of FIG. 29, the electronic apparatus 100 displays the first screen 2931 including the first body part UI (2932, 2932) for a first user. However, a second user, not the first user, may be standing on the electronic apparatus 100. The electronic apparatus 100 may acquire the touch data for the second user.

The electronic apparatus 100 may identify that the first user authentication has failed based on the touch data of the second user, not the first user. When the first user authentication has failed, the electronic apparatus 100 may provide UIs 3011, 3012, 3013, and 3014 to notify that the first user authentication has failed.

The UI 3011 may include text information indicating that the first user authentication has failed.

UIs 3012 and 3013 may be a UI that changes the color of the first body part UIs 2932 and 2933 included in the first screen 2931 in the embodiment 2930 of FIG. 29 differently.

A UI 3014 may indicate an unregistered user.

Referring to embodiment 3020 of FIG. 30, the electronic apparatus 100 may perform user authentication for a second user. The electronic apparatus 100 may re-perform the first user authentication for the second user who is a new user. The electronic apparatus 100 may perform the first user authentication based on the touch data input by the second user. When performing the first user authentication for a new user, the electronic apparatus 100 may provide UIs 3021 and 3022 to notify re-search.

For example, the first user authentication may use account information by device connection. It is assumed that a terminal apparatus of a second user exists. The electronic apparatus 100 may request account information from the terminal apparatus of the second user. The terminal apparatus of the second user may transmit the account information to the electronic apparatus 100 in response to the request. The electronic apparatus 100 may identify whether the account information of the second user is already registered. When the account information is already registered, the electronic apparatus 100 may identify that the first user authentication for the second user is successful.

For example, the first user authentication may use the audio data. The electronic apparatus 100 may request a voice input from the second user. When a voice input is received from the second user, the electronic apparatus 100 may acquire the audio data of the second user. The electronic apparatus 100 may perform the first user authentication for the second user based on the audio data.

Referring to the embodiment 3030 of FIG. 30, it is assumed that the touch data for the second user matches the registered touch data. It is assumed that the second user is a user who has been previously registered with the electronic apparatus 100. When the first user authentication is completed for the second user, the electronic apparatus 100 may provide UIs 3031 and 3032 to notify that the first user authentication is successful.

A UI 3021 may indicate that authentication is successful.

UI 3022 may indicate the target (second user) for which authentication is successful.

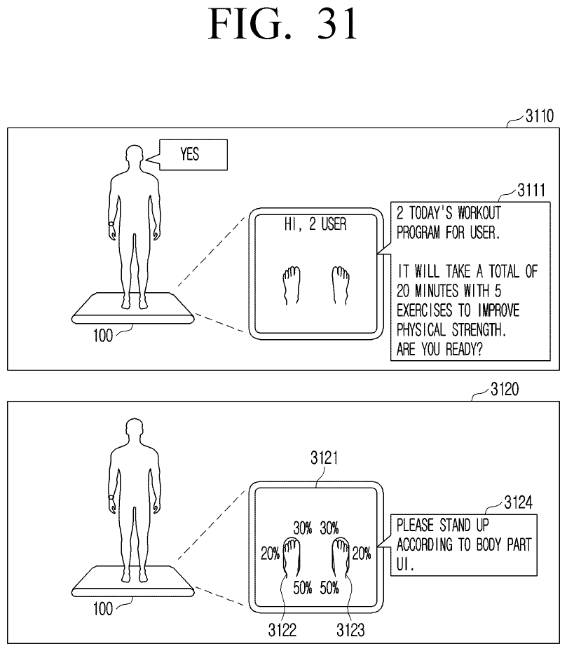

FIG. 31 is a diagram for describing an embodiment in which a user is replaced according to an embodiment.

Referring to an embodiment 3110 of FIG. 31, after the first user authentication for the second user is successful according to embodiment 3030 of FIG. 30, the electronic apparatus 100 may provide UI 3111 to suggest the target exercise. As an example, the electronic apparatus 100 may provide UI 3111 including “5 exercises for physical strength enhancement, a total of 20 minutes. Are you ready?” The UI 3111 may be provided as at least one of image data or the audio data. In response to the provided UI 3111, the user may input a control command requesting the start of the exercise content. The electronic apparatus 100 may receive a control command (or user input) requesting the start of the exercise content from the user.

Referring to the embodiment 3120 of FIG. 31, after the control command requesting the start of the exercise content is received, the electronic apparatus 100 may display the first screen 3121 for providing the exercise content. The first screen 3121 may include first body part UIs 3122 and 3123. The second user may touch a body part of the second user to the electronic apparatus 100 through the first body part UIs 3122 and 3123. The electronic apparatus 100 may provide a UI 3124 requesting a specific posture for the second user authentication. As an example, the UI 3124 may include “Please stand up according to the body part UI.”

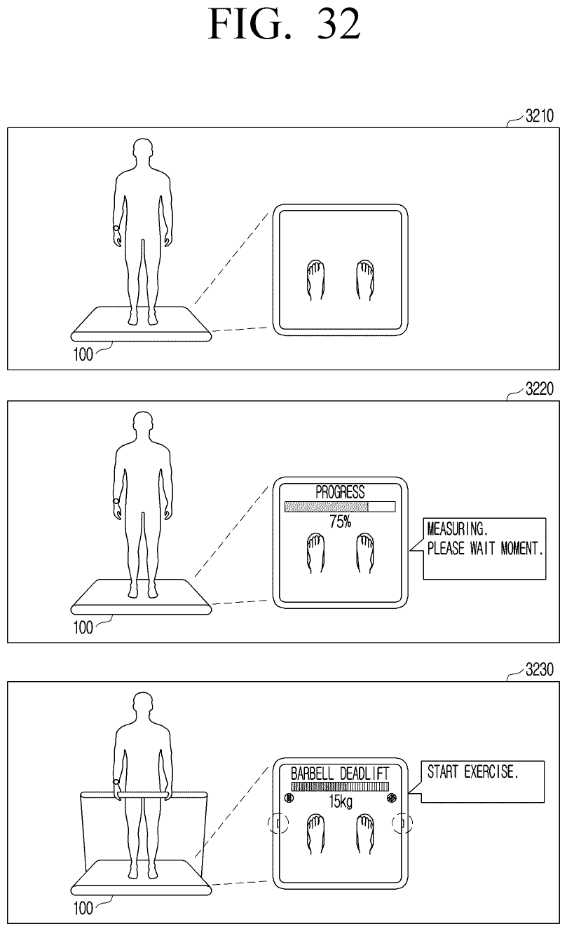

An embodiment 3120 of FIG. 31 may be followed by the embodiment 3210 of FIG. 32.

FIG. 32 is a diagram for describing an embodiment in which a user is replaced according to an embodiment. The user being replaced may refer to a scenario where after a first user is authenticated and completes one or more exercises, the first user leaves the electronic apparatus and a second user is authenticated for the electronic apparatus.

An embodiment 3210 of FIG. 32 may correspond to the embodiment 2610 of FIG. 26. The embodiment 3220 of FIG. 32 may correspond to the embodiment 2620 of FIG. 26. The embodiment 2630 of FIG. 32 may correspond to the embodiment 2630 of FIG. 26. Duplicate descriptions are omitted.

The target exercise of the embodiment of FIG. 26 may be different from the target exercise of FIG. 32. Some the information related to the target exercise may be different.

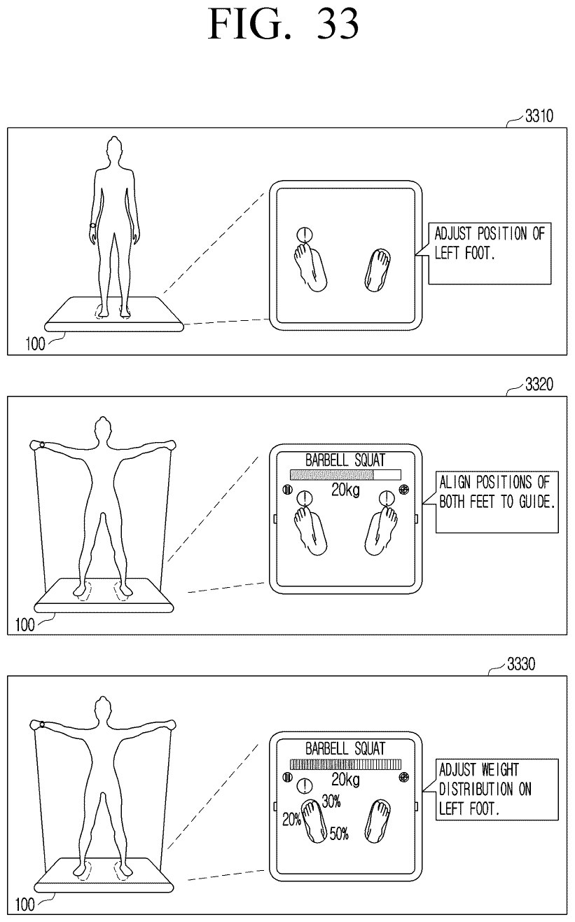

FIG. 33 is a diagram for describing an operation of providing a feedback UI according to an embodiment.

Referring to an embodiment 3310 of FIG. 33, the electronic apparatus 100 may provide feedback while performing the second user authentication. The electronic apparatus 100 may acquire an average difference between the location of the first body part UI displayed for the second user authentication and the user touch location. When the average difference is greater than or equal to a threshold value, the electronic apparatus 100 may provide feedback for posture correction. For example, the feedback may include the content “Please adjust the left foot position.” For example, the feedback may include an image representing posture correction.

Referring to the embodiment (3320) of FIG. 33, the electronic apparatus 100 may provide feedback while providing the exercise content. The electronic apparatus 100 may display the second body part UI to guide the posture of the target exercise. The electronic apparatus 100 may acquire an average difference between the location of the second body part UI and the user touch location. When the average difference is greater than or equal to a threshold value, the electronic apparatus 100 may provide feedback for posture correction. For example, the feedback may include the content “Please adjust the position of both feet.” For example, the feedback may include an image indicating posture correction.

Referring to an embodiment 3330 of FIG. 33, the electronic apparatus 100 may provide feedback for correcting pressure distribution while providing the exercise content. The electronic apparatus 100 may display a UI indicating pressure distribution. The electronic apparatus 100 may acquire an average difference value between a preset pressure distribution and a measured pressure distribution. When the average difference value is greater than or equal to a threshold value, the electronic apparatus 100 may provide feedback for posture correction. For example, the feedback may include the content “Please adjust the weight distribution of the left foot.” For example, the feedback may include an image indicating the correction of pressure distribution.

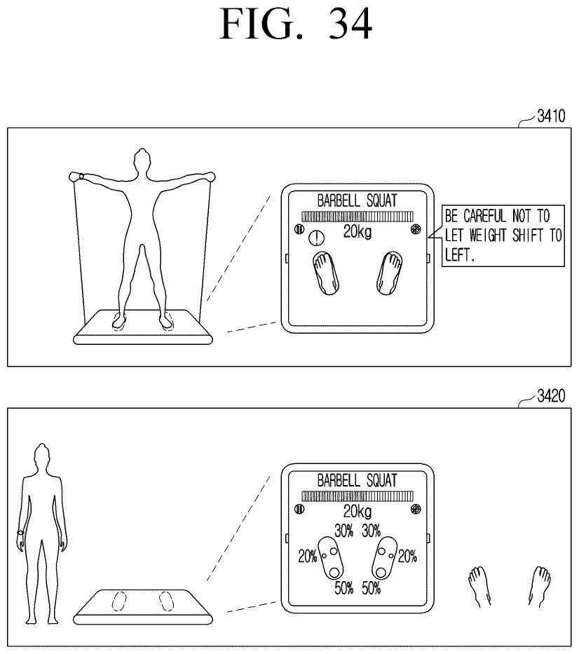

FIG. 34 is an operation for explaining an operation of providing a feedback UI according to one embodiment.

Referring to an embodiment 3410 of FIG. 34, the electronic apparatus 100 may provide feedback indicating a risk of injury. When the pressure of the left foot is greater than the pressure of the right foot by a threshold value or more, the electronic apparatus 100 may provide feedback indicating a risk of injury. For example, the feedback may include “Please be careful not to let your weight shift to the left.”

Referring to an embodiment 3420 of FIG. 34, it is assumed that the user gets off the electronic apparatus 100 based on the feedback provided in the embodiment 3410. When the electronic apparatus 100 identifies that the user has gotten off, the electronic apparatus 100 may display a screen for the exercise content again.

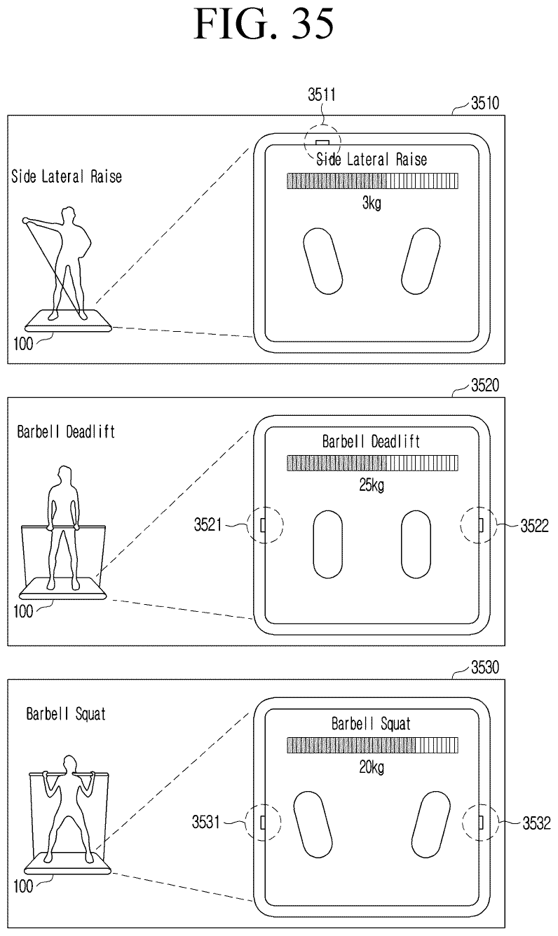

FIG. 35 is a diagram for describing an operation of displaying a UI indicating a cable position according to one embodiment.

Referring to the embodiment 3510 of FIG. 35, the electronic apparatus 100 may provide the exercise content for the target exercise (side lateral raise). The electronic apparatus 100 may provide a UI 3511 indicating a position of a cable corresponding to the target exercise (side lateral raise).

Referring to the embodiment 3520 of FIG. 35, the electronic apparatus 100 may provide the exercise content for the target exercise (barbell deadlift). The electronic apparatus 100 may provide a UI 3521 and 3522 indicating the position of a cable corresponding to the target exercise (barbell deadlift).

Referring to the embodiment 3530 of FIG. 35, the electronic apparatus 100 may provide the exercise content for the target exercise (barbell squat). The electronic apparatus 100 may provide a UI 3531 and 3532 indicating the position of a cable corresponding to the target exercise (barbell squat).

The location of the cable may be different depending on the target exercise. When the cable location is different, the position of the UI indicating the cable location may be different.

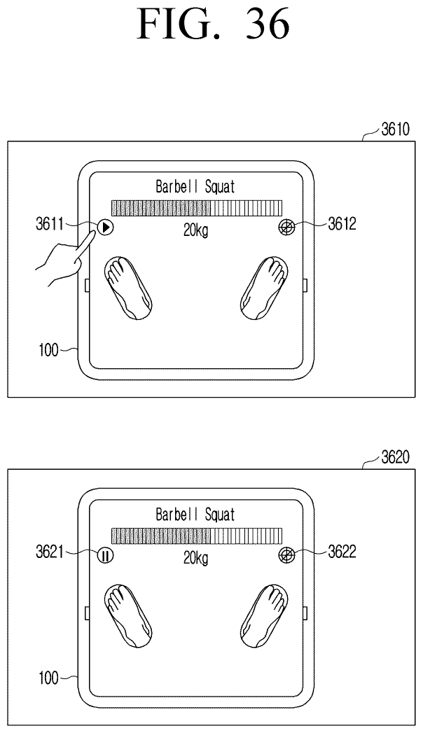

FIG. 36 is a diagram for describing an operation of providing the exercise content according to an embodiment.

Referring to the embodiment 3610 of FIG. 36, the electronic apparatus 100 may be in a state where the exercise content is stopped. When a user input for selecting the UI 3611 is received, the electronic apparatus 100 may play the exercise content.

Referring to the embodiment 3620 of FIG. 36, the electronic apparatus 100 may be in a state where the exercise content is being played. When a user input for selecting the UI 3621 is received, the electronic apparatus 100 may play the exercise content.

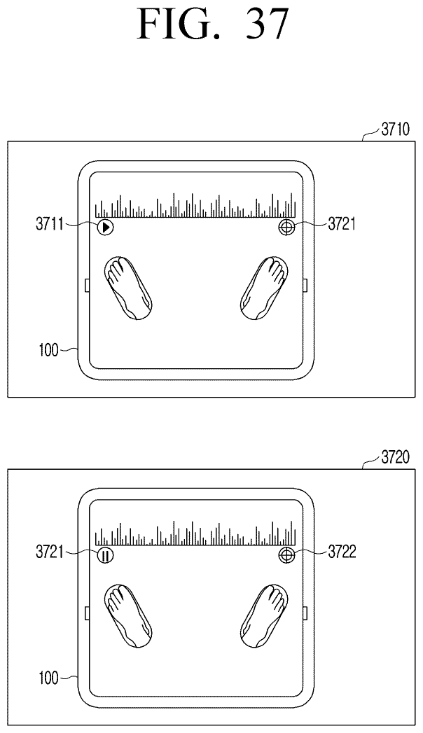

The UI 3612 and 3622 may be a UI for not displaying guide information related to the exercise content (the information related to the target exercise). In the embodiments 3610 and 3620, it is assumed that the guide information is being displayed. When a user input for selecting the UI 3612 and 3622 is received, the electronic apparatus 100 may not display the guide information. A description related to this is described in FIG. 37.

FIG. 37 is a diagram for describing an operation of providing guide information related to the exercise content according to an embodiment.

Referring to an embodiment 3710 of FIG. 37, the electronic apparatus 100 may be in a state of stopping the exercise content. When a user input of selecting UI 3711 is received, the electronic apparatus 100 may play the exercise content.

Referring to an embodiment 3720 of FIG. 37, the electronic apparatus 100 may be in a state of playing the exercise content. When a user input of selecting UI 3721 is received, the electronic apparatus 100 may play the exercise content.

In the embodiments 3710 and 3720, the electronic apparatus 100 may provide the exercise content without displaying guide information.

UIs 3712 and 3722 may be a UI for displaying guide information related to the exercise content (information related to target exercise). In the embodiments 3710 and 3720, it is assumed that the guide information is not displayed. When a user input for selecting UI 3712 and 3722 is received, the electronic apparatus 100 may display the guide information.

In FIG. 37, the exercise content may be replaced with music content. When music content is provided, the electronic apparatus 100 may provide a visual effect corresponding to the music content.

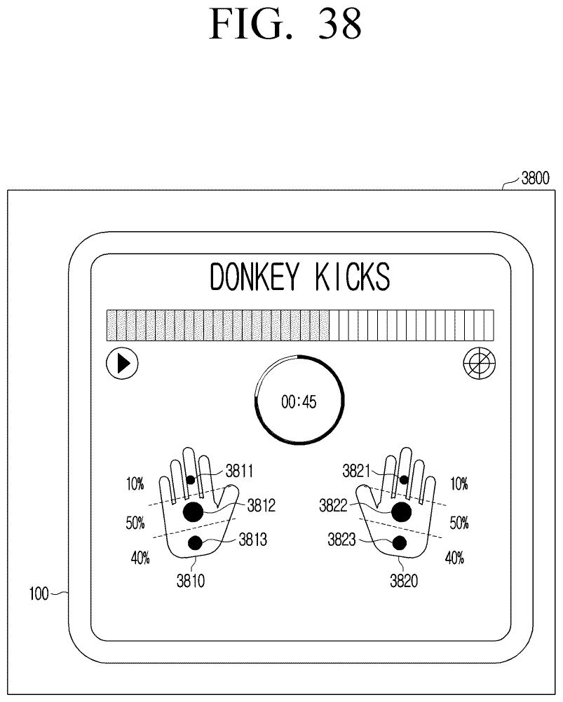

FIG. 38 is a diagram for describing a screen related to the exercise content according to an embodiment.

Referring to FIG. 38, the electronic apparatus 100 may display a screen for the exercise content. The electronic apparatus 100 may display the second body part UI 3810 and 3820. The electronic apparatus 100 may provide a UI 3811, 3812, 3813, 3821, 3822, and 3823 indicating pressure distribution together with the second body part UI 3810 and 3820. Through the UI 3811, 3812, 3813, 3821, 3822, and 3823 indicating pressure distribution, the user may easily determine the degree of force distribution.

FIG. 39 is a diagram for describing the body part UI corresponding to exercise according to an embodiment.

Referring to the embodiment 3910 of FIG. 39, the electronic apparatus 100 may provide the second body part UI 3911 corresponding to the target exercise (plank bird dog).

Referring to an embodiment 3920 of FIG. 39, the electronic apparatus 100 may provide the second body part UI 3921 corresponding to the target exercise (plank kick throughs).

Referring to an embodiment 3930 of FIG. 39, the electronic apparatus 100 may provide the second body part UI 3931 corresponding to the target exercise (triangle crunch).

Referring to the embodiment 3940 of FIG. 39, the electronic apparatus 100 may provide the second body part UI 3941 corresponding to the target exercise (side plank rotation).

The body part may be different for each target exercise. The body part may be a part that comes into contact with the electronic apparatus 100 in the posture of the target exercise.

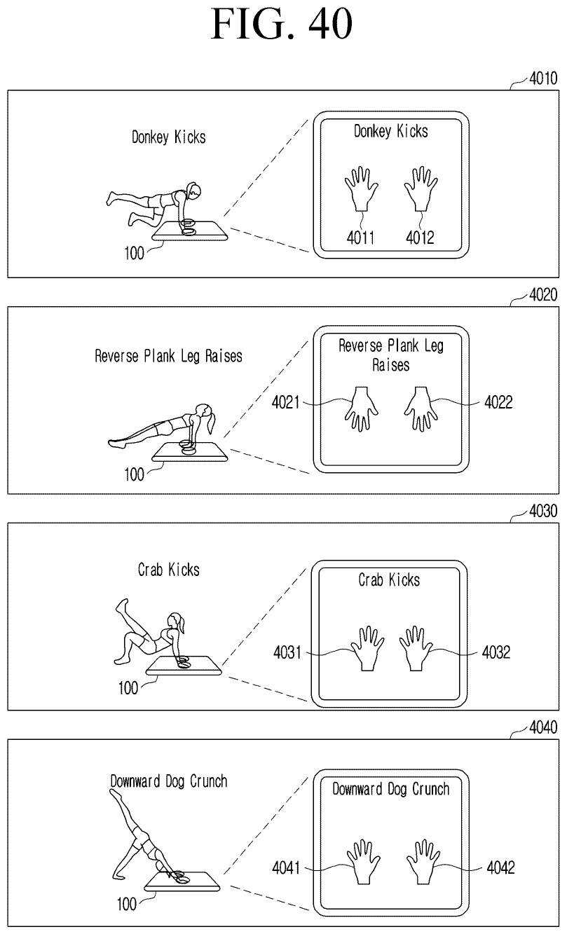

FIG. 40 is a diagram for describing the body part UI corresponding to exercise according to an embodiment.

Referring to an embodiment 4010 of FIG. 40, the electronic apparatus 100 may provide the second body part UI 4011 and 4012 corresponding to the target exercise (donkey kicks).

Referring to the embodiment 4020 of FIG. 40, the electronic apparatus 100 may provide the second body part UI 4021 and 4022 corresponding to the target exercise (reverse plank leg raises).

Referring to the embodiment 4030 of FIG. 40, the electronic apparatus 100 may provide the second body part UI 4031 and 4032 corresponding to the target exercise (crab kicks).

Referring to the embodiment 4040 of FIG. 40, the electronic apparatus 100 may provide the second body part UI 4041 and 4042 corresponding to the target exercise (downward dog crunch).

The second body part UI may be plural depending on the target exercise. Even when the body part is the same, the direction and arrangement position may be different depending on the target exercise.

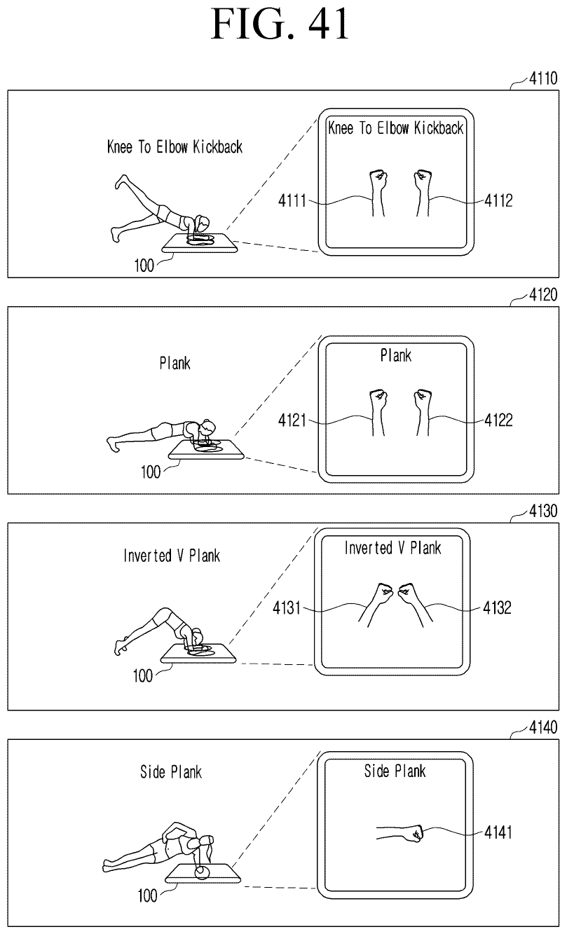

FIG. 41 is a diagram for describing the body part UI corresponding to exercise according to an embodiment.

Referring to the embodiment 4110 of FIG. 41, the electronic apparatus 100 may provide the second body part UI 4111 and 4112 corresponding to the target exercise (knee to elbow kickback).

Referring to the embodiment 4120 of FIG. 41, the electronic apparatus 100 may provide the second body parts UI 4121 and 4122 corresponding to the target exercise (plank).

Referring to the embodiment 4130 of FIG. 41, the electronic apparatus 100 may provide the second body parts UI 4131 and 4132 corresponding to the target exercise (inverted V plank).

Referring to the embodiment 4140 of FIG. 41, the electronic apparatus 100 may provide the second body part UI 4141 corresponding to the target exercise (side plank).

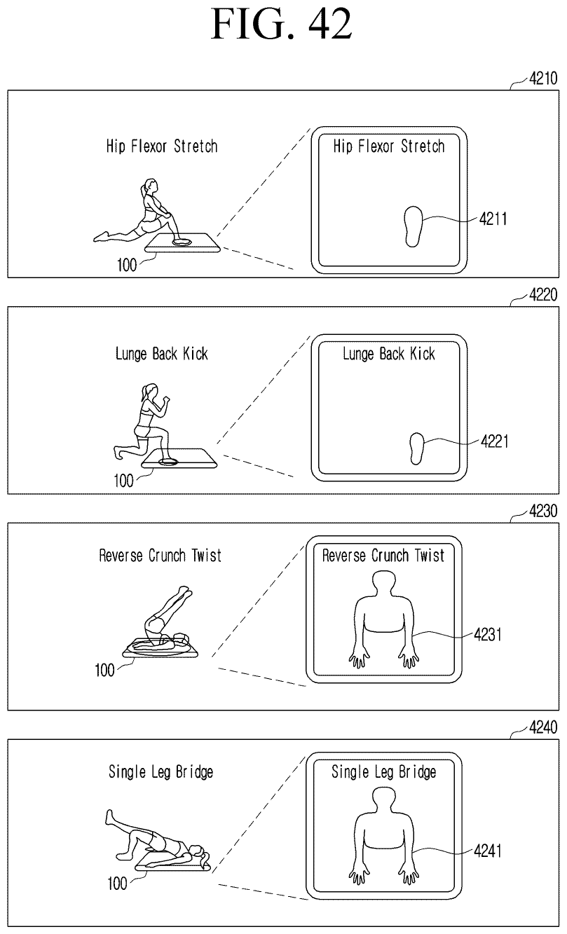

FIG. 42 is a diagram for describing the body part UI corresponding to exercise according to an embodiment.

Referring to an embodiment 4210 of FIG. 42, the electronic apparatus 100 may provide the second body part UI 4211 corresponding to the target exercise (hip flexor stretch).

Referring to an embodiment 4220 of FIG. 42, the electronic apparatus 100 may provide the second body part UI 4221 corresponding to the target exercise (lunge back kick).

Referring to an embodiment 4230 of FIG. 42, the electronic apparatus 100 may provide the second body part UI 4231 corresponding to the target exercise (reverse crunch twist).

Referring to the embodiment 4240 of FIG. 42, the electronic apparatus 100 may provide the second body part UI 4241 corresponding to the target exercise (single leg bridge).

The body part may not represent only one part of a body, but may represent a body shape that is in contact with the electronic apparatus 100. Therefore, the body part may not be specified. The second body part UI may be displayed based on a body shape that is to be in contact with the electronic apparatus 100.

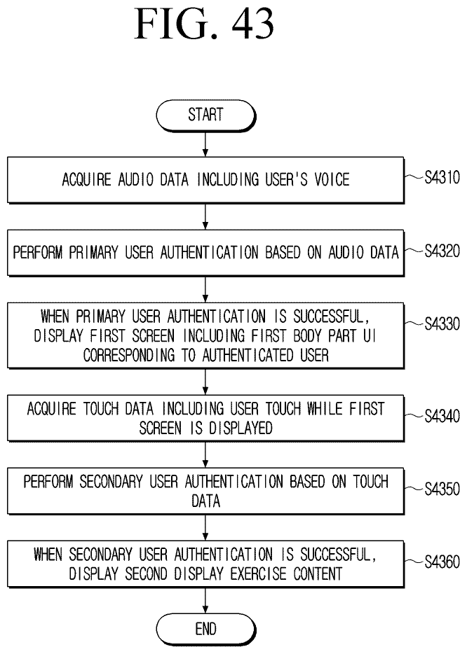

FIG. 43 is a diagram for describing a control operation of an electronic apparatus according to an embodiment.

Referring to FIG. 43, a control method of an electronic apparatus includes an operation of acquiring the audio data including a user's voice (S4310), an operation of performing the first user authentication based on the audio data (S4320), an operation of displaying the first screen including the first body part UI corresponding to an authenticated user when the first user authentication is successful (S4330), an operation of acquiring the touch data including the user touch while the first screen is displayed (S4340), an operation of performing the second user authentication based on the touch data (S4350), and an operation of displaying the second screen for the exercise content when the second user authentication is successful (S4360).

The operation of performing the first user authentication (S4320) may acquire a first difference value between the audio data and registered voice data stored in the electronic apparatus, and when the first difference value is less than a first threshold value, it may be identified that the first user authentication is successful.

The operation of displaying the first screen (S4330) may include, when the first user authentication is identified as successful, acquiring the body part information corresponding to the authenticated user, identifying the preset first target part among a plurality of body parts included in the body part information, generating the first body part UI based on the first target part, generating the first screen including the first body part UI, and displaying the first screen.

The control method may include, when the first screen is displayed, activating the sensor unit that acquires the touch data.

The operation of performing the second user authentication (S4350) may include acquiring the second difference value between the touch data and the registered touch data stored in the electronic apparatus, and when the difference value is less than the second threshold value, identifying the second user authentication as successful.

The operation of displaying the second screen (S4360) may include, when the second user authentication is identified as successful, acquiring the user information corresponding to the authenticated user, generating the second screen based on the user information, and displaying the second screen.

The user information may include at least one of the body part information, the body analysis information, the schedule information, the preferred exercise information, or the history information.

The operation of displaying the second screen (S4360) may determine the target exercise among a plurality of exercises based on the user information, and generate the second screen for guiding the target exercise.

The operation of displaying the second screen (S4360) may identify a posture of the target exercise when the target exercise is determined, identify a second target part of the authenticated user who should touch the electronic apparatus based on the posture of the target exercise, and a touch location of the second target part, and generate the second body part UI based on the information corresponding to the second target part among the body part information and the touch location, and generate the second screen including the second body part UI, and display the second screen.

The control method may include an operation of identifying the cable location corresponding to the target exercise based on the cable location table stored in the electronic apparatus when a grip is required in the target exercise, and an operation of providing the UI indicating the cable location.

The above-described methods according to various embodiments of the disclosure may be implemented in a form of application that may be installed in the existing electronic apparatus.

The above-described methods according to various embodiments of the disclosure may be implemented only by software upgrade or hardware upgrade of the existing electronic apparatus.

Various embodiments of the disclosure described above may also be performed through an embedded server included in the electronic apparatus or an external server of at least one of the electronic apparatus or the display apparatus.

According to an embodiment of the disclosure, various embodiments described above may be implemented by software including instructions stored in a machine-readable storage medium (for example, a computer-readable storage medium). A machine is an apparatus capable of calling a stored instruction from a storage medium and operating according to the called instruction, and may include the electronic apparatus of the embodiments disclosed. In the case in which the above-described command is executed by the processor, the processor may directly perform a function corresponding to the command or other components may perform the function corresponding to the command under a control of the processor. The command may include codes created or executed by a compiler or an interpreter. The machine-readable storage medium may be provided in a form of a non-transitory storage medium. Here, the term “non-transitory” means that the storage medium is tangible without including a signal, and does not distinguish whether data are semi-permanently or temporarily stored in the storage medium.

According to an embodiment of the disclosure, the above-described methods according to the diverse embodiments may be included and provided in a computer program product. The computer program product may be traded as a product between a seller and a purchaser. The computer program product may be distributed in a form of a storage medium (for example, a compact disc read only memory (CD-ROM)) that may be read by the machine or online through an application store. In case of the online distribution, at least a portion of the computer program product may be at least temporarily stored in storage media such as a memory of a server of a manufacturer, a server of an application store, or a relay server or be temporarily generated.

Each of components (for example, modules or programs) according to various embodiments described above may include a single entity or a plurality of entities, and some of the corresponding sub-components described above may be omitted or other sub-components may be further included in the diverse embodiments. Alternatively or additionally, some components (e.g., modules or programs) may be integrated into one entity and perform the same or similar functions performed by each corresponding component prior to integration. Operations performed by the modules, the programs, or the other components according to the diverse embodiments may be executed in a sequential manner, a parallel manner, an iterative manner, or a heuristic manner, at least some of the operations may be performed in a different order or be omitted, or other operations may be added.

Although embodiments of the disclosure have been illustrated and described hereinabove, the disclosure is not limited to the above-described specific embodiments, but may be variously modified by those skilled in the art to which the disclosure pertains without departing from the gist of the disclosure as disclosed in the accompanying claims. These modifications should also be understood to fall within the spirit of the disclosure.

What is claimed is:

1. An electronic apparatus comprising:
a memory configured to store instructions;
a microphone;
a sensor;
a display; and
at least one processor including processing circuitry,
wherein the instructions, when executed by the at least one processor individually or collectively, cause the electronic apparatus to:
acquire, through the microphone, audio data including a voice of a user,
perform a first user authentication based on the audio data,
based on identifying that the first user authentication is successful, control the display to display a first screen including a first body part user interface (UI) corresponding to an authenticated user,
acquire, through the sensor, touch data including a user touch while the first screen is displayed,
perform second user authentication based on the touch data, and
based on identifying that the second user authentication is successful, control the display to display a second screen for exercise content.

2. The electronic apparatus as claimed in claim 1, wherein the instructions, when executed by the at least one processor individually or collectively, cause the electronic apparatus to:
acquire a first difference value between the audio data acquired through the microphone and registered voice data stored in the memory, and
identify that the first user authentication is successful based on the first difference value being less than a first threshold value.

3. The electronic apparatus as claimed in claim 1, wherein the instructions, when executed by the at least one processor individually or collectively, cause the electronic apparatus to:
based on identifying that the first user authentication is successful, acquire body part information corresponding to the authenticated user,
identify a preset first target part among a plurality of body parts included in the body part information,
generate the first body part UI based on the preset first target part,
generate the first screen including the first body part UI, and
control the display to display the first screen.

4. The electronic apparatus as claimed in claim 3, wherein the instructions, when executed by the at least one processor individually or collectively, cause the electronic apparatus to:
based on the first screen being displayed, activate the sensor.

5. The electronic apparatus as claimed in claim 1, wherein the instructions, when executed by the at least one processor individually or collectively, cause the electronic apparatus to:
acquire a second difference value between the touch data acquired through the sensor and registered touch data stored in the memory, and
identify that the second user authentication is successful based on the second difference value being less than a second threshold value.

6. The electronic apparatus as claimed in claim 1, wherein the instructions, when executed by the at least one processor individually or collectively, cause the electronic apparatus to:
based on identifying that the second user authentication is successful, acquire user information corresponding to the authenticated user,
generate the second screen based on the user information, and
control the display to display the second screen.

7. The electronic apparatus as claimed in claim 6, wherein the user information includes at least one of body part information, body analysis information, schedule information, preferred exercise information, or history information.

8. The electronic apparatus as claimed in claim 7, wherein the instructions, when executed by the at least one processor individually or collectively, cause the electronic apparatus to:
determine a target exercise among a plurality of exercises based on the user information, and
generate the second screen providing guidance for the target exercise.

9. The electronic apparatus as claimed in claim 8, wherein the instructions, when executed by the at least one processor individually or collectively, cause the electronic apparatus to:
identify a posture of the target exercise based on determining the target exercise,
identify a second target part of the authenticated user based on the posture of the target exercise and a touch location of the second target part on the electronic apparatus,
generate a second body part UI based on information corresponding to the second target part among the body part information and the touch location,
generate the second screen including the second body part UI, and
control the display to display the second screen.

10. The electronic apparatus as claimed in claim 8, wherein the instructions, when executed by the at least one processor individually or collectively, cause the electronic apparatus to:
identify a cable location corresponding to the target exercise based on a cable location table stored in the memory when a grip is required in the target exercise, and
provide a UI indicating the cable location.

11. A controlling method of an electronic apparatus, the controlling method comprising:
acquiring audio data including a voice of a user;
performing a first user authentication based on the audio data;
based on identifying that the first user authentication is successful, displaying a first screen including a first body part user interface (UI) corresponding to an authenticated user;
acquiring touch data including a user touch while the first screen is displayed;
performing second user authentication based on the touch data; and
based on identifying that the second user authentication is successful, displaying a second screen for exercise content.

12. The controlling method as claimed in claim 11, wherein the performing the first user authentication comprises:
acquiring a first difference value between the audio data and registered voice data stored in the electronic apparatus, and
identifying that the first user authentication is successful based on the first difference value being less than a first threshold value.

13. The controlling method as claimed in claim 11, wherein the displaying the first screen further comprises:
based on identifying the first user authentication is successful, acquiring body part information corresponding to the authenticated user;
identifying a preset first target part among a plurality of body parts included in the body part information;
generating the first body part UI based on the preset first target part;
generating the first screen including the first body part UI; and
displaying the first screen.

14. The controlling method as claimed in claim 13, further comprising:
based on the first screen being displayed, activating a sensor for acquiring the touch data.

15. The controlling method as claimed in claim 11, wherein the performing the second user authentication comprises:
acquiring a second difference value between the touch data and registered touch data stored in the electronic apparatus; and
identifying that the second user authentication is successful based on the second difference value being less than a second threshold value.

16. A non-transitory computer readable medium having instructions stored therein, which when executed by a processor in an electronic apparatus cause the electronic apparatus to execute a method comprising:
acquiring audio data including a voice of a user;
performing a first user authentication based on the audio data;
based on identifying that the first user authentication is successful, displaying a first screen including a first body part user interface (UI) corresponding to an authenticated user;
acquiring touch data including a user touch while the first screen is displayed;
performing second user authentication based on the touch data; and
based on identifying that the second user authentication is successful, displaying a second screen for exercise content.

17. The non-transitory computer readable medium according to claim 16, wherein the performing the first user authentication comprises:
acquiring a first difference value between the audio data and registered voice data stored in the electronic device, and
identify that the first user authentication is successful based on the first difference value being less than a first threshold value.

18. The non-transitory computer readable medium according to claim 16, wherein the displaying the first screen comprises:
based on identifying that the first user authentication is successful, acquiring body part information corresponding to the authenticated user;
identifying a preset first target part among a plurality of body parts included in the body part information;
generating the first body part UI based on the preset first target part;
generating the first screen including the first body part UI; and
displaying the first screen.

19. The non-transitory computer readable medium according to claim 18, further comprising:
based on determining that the first screen is displayed, activating the sensor circuitry for acquiring the touch data based.

20. The non-transitory computer readable medium according to claim 16, wherein the performing the second user authentication comprises:
acquiring a second difference value between the touch data and registered touch data stored in the electronic device; and
identifying that the second user authentication is successful based on the second difference value being less than a second threshold value.
